(19) United States
(12) Patent Application Publication
(10) Pub. No.: US 2008/0108894 A1
(43) Pub. Date: May 8, 2008

(54) Methods and Systems of Analyzing Clinical Parameters and Methods of Producing Visual Images

(76) Inventors: Gabriel A. Elgavish, Hoover, AL (US); Pal Suranyi, Birmingham, AL (US); Tamar Simor, Pecs (HU); Pal P. Kiss, Vestavia Hills, AL (US)

Correspondence Address: THOMAS, KAYDEN, HORSTEMEYER & RISLEY, LLP, 600 GALLERIA PARKWAY, S.E., STE 1500, ATLANTA, GA, 30339-5994, US

(21) Appl. No.: 11/718,775

(22) PCT Filed: Nov. 15, 2005
(86) PCT No.: PCT/US05/41203

§ 371 Date: May 7, 2007

Related U.S. Application Data
(60) Provisional application No. 60/628,037, filed on Nov. 15, 2004.

(51) Int. Cl. A61B 5/055; A61B 5/00

(52) U.S. Cl. 600/420; 600/300; 600/407; 600/410

(57) ABSTRACT

Methods of analyzing non-clinical parameters, methods of analyzing clinical parameters, methods of producing visual images, systems of analyzing non-clinical parameters, systems of analyzing clinical parameters, systems of producing visual images, systems of producing reports, reports based on imaging modality information, and displays based on imaging modality information, are disclosed.

### CROSS-REFERENCE TO RELATED APPLICATION

This application claims priority to U.S. provisional application entitled, “Differentiation of tissue clinical parameters using nuclear magnetic resonance techniques,” having Ser. No. 60/628,037, filed on Nov. 15, 2004, which is entirely incorporated herein by reference.

### TECHNICAL FIELD

The present disclosure is generally related to imaging and, more particularly, is related to using magnetic resonance imaging.

### BACKGROUND

Numerous methods have been developed to localize and visualize tissues damaged as a result of a vascular occlusion event and the resulting myocardial ischemia. Non-contrast MRI approaches have been used to provide some information regarding the extent of myocardial injury. This is possible since areas of acute myocardial damage (i.e., infarcts) show a hyperintense signal on T2-weighted spin-echo images. However, these approaches may overestimate acute infarct size by including area at, risk and they do not identify chronic infarcts. Also, T2-weighted images often have a low signal-to-noise ratio.

Among non-MRI techniques SPECT with nuclear imaging (e.g., with 99 mTc-sestamibi) has been used in the clinic to determine infarct size. Although providing some useful information, this technique suffers from two limitations: 1) the effects of scatter; and 2) attenuation and partial volume effects related to severely abnormal wall motion. Stress- and contrast-echo also have significant limitations.

Contrast-enhanced MRI using Delayed Enhancement (ceMRI with DE) has been shown to be a superior approach to MRI determination of infarct size and assessment of recovery of function compared to the techniques discussed above. The effectiveness of ceMRI DE is due to the accumulation of the contrast agent into irreversibly injured areas of the myocardium. However, this method does not yield accurate and reproducible information on the density of the myocardial infarct highlighted. The enhancement of the MRI signal intensity in a given myocardial area depends on the concentration of the contrast agent in the myocardial volume element represented by the area in question. As the concentration of contrast agent increases in a given myocardial volume element, the MRI signal increases in intensity. This increased MRI signal (referred to as the SIE, or signal intensity enhancement) is measured. The ability to accurately and reproducibly quantify SIE, however, is impaired by several factors. The intrinsic physical parameter that is enhanced in linear proportion with the contrast agent concentration is the paramagnetic relaxation rate enhancement, ΔR1. ΔR1 is the difference between the inverse T1 with and without the presence of contrast agent in ceMRI. The SIE itself is dependent on this ΔR1, but not in a linear fashion.

Furthermore, SIE is not an intrinsic MRI parameter. This parameter is always dependent on the particular set of MRI acquisition parameters and the pulse sequence used. Another confounding factor is the ever present inhomogeneity of the magnetic field of the MRI transceiver coil, artificially imparting varying signal intensities to different parts of the heart depending on their distance from, and relative position to, the coil (referred to as the coil-effect).

In short, a consistent and accurate determination of contrast agent concentration and distribution, and thus extent of myocardial damage/infarction, over the entire heart and over different times (let alone different MRI sessions or instruments) is fraught with difficulty when using SIE as the MRI parameter being measured.

Researchers have used contrast echocardiography in human patients to determine myocardial viability in acute infarction and to predict recovery of resting function and contractile reserve. It was demonstrated that even when resting function does not recover, partial viability could still be beneficial for contractile reserve, exercise tolerance, prevention of remodeling, maintaining LV shape, and improving survival after acute myocardial infarction (MI). It was found that in regions with persistent, severe wall motion abnormalities, the presence of patchy epicardial viability identifies regions with contractile reserve. Although mid- and epicardial portions of the left ventricle contribute little to resting function, they become important for augmentation of systolic performance during exertion. The exact viability threshold for the possibility of recovery of function of any myocardial volume element is not known. It is clear, however, that the lower the extent of infarct of any such volume element, the higher its potential for ultimate recovery.

Triphenyltetrazolium-Chloride (TTC) staining is a well-established research tool and is used to determine infarct size and potentially percent-infarct with good resolution. Unfortunately, TTC staining can be done only once, and only on excised hearts, and thus it is completely irrelevant to the clinical context other than as a post-mortem technique. It would be highly desirable to possess a method that were at least as quantitative as TTC staining, which yields accurate and reproducible results but one that could be carried out in vivo, and repeatedly if so desired.

In summary, the art at present is lacking a method for accurately and reproducibly determining a variety of tissue clinical parameters such that this information could be used by clinicians. Accordingly, there is a need in the industry to address the aforementioned deficiencies and/or inadequacies.

### SUMMARY

Methods of analyzing non-clinical parameters, methods of analyzing clinical parameters, methods of producing visual images, systems of analyzing non-clinical parameters, systems of analyzing clinical parameters, systems of producing visual images, systems of producing reports, reports based on imaging modality information, and displays based on imaging modality information, are disclosed.

An embodiment of a method, among others, includes: obtaining an imaging modality parameter for a plurality of select regions of a structure, wherein each of the measurements of the imaging modality parameter is a partial volume average value of the imaging modality parameter for each of the select regions; processing the imaging modality parameters for each of the select regions to produce a data set; and converting the data set into a format to convey information of the data set.

An embodiment of a method, among others, includes: obtaining a magnetic resonance physical parameter selected from, but not limited to, one of R1, R2, R2*, signal intensity (SI), signal intensity at equilibrium (SI₀), proton density (PD), and combinations thereof, for a plurality of select regions of a tissue, wherein R1 is a longitudinal relaxation rate parameter, R2 is the irrecoverable transverse relaxation rate parameter, and R2* the total transverse relaxation rate parameter; processing the magnetic resonance parameter for each of the select regions; and forming a visual display of the tissue sample from the magnetic resonance physical parameters.

An embodiment of a display, among others, includes: a visual display formed by one of the methods described herein.

An embodiment of a computer system, among others, includes: a system that implements the methods described herein, and a display device that displays a visual display formed using the methods.

An embodiment of a display, among others, includes: a visual quantitative display of a tissue sample, wherein the visual display is formed from at least one magnetic resonance physical parameter obtained for each of a set of select regions of the tissue sample, wherein magnetic resonance physical parameter selected from, but not limited to, one of R1, R2, R2*, signal intensity (SI), signal intensity at equilibrium (SI₀), proton density (PD), and combinations thereof, wherein R1 is a longitudinal relaxation rate parameter, R2 is the irrecoverable transverse relaxation rate parameter, and R2* is the experimentally observed transverse relaxation rate parameter.

An embodiment of a method of evaluating a condition, among others, includes: generating a visual display as described herein; and evaluating a clinical parameter illustrated in the visual display, wherein the clinical parameter corresponds to a condition in a tissue.

An embodiment of a display, among others, includes: a formatted data set in a format selected from one of the following: a visual display, a report, a media used to convey information, and a combination thereof, wherein the data set is generated from an imaging modality parameter for a plurality of select regions of a structure, wherein each of the measurements of the imaging modality parameter is a partial volume average value of the imaging modality parameter for each of the select regions.

Other systems, methods, features, and advantages of the present disclosure will be, or become, apparent to one with skill in the art upon examination of the following drawings and detailed description. It is intended that all such additional systems, methods, features, and advantages be included within this description, be within the scope of the present disclosure, and be protected by the accompanying claims.

### BRIEF DESCRIPTION OF THE DRAWINGS

Many aspects of the disclosed systems and methods can be better understood with reference to the following drawings. The components in the drawings are not necessarily to scale, emphasis instead being placed upon clearly illustrating the relevant principles. Moreover, in the drawings, like reference numerals designate corresponding parts throughout the several views.

FIG. 1 is an illustrative embodiment of a flow chart of a calculation of a percent map.

FIG. 2A illustrates voxel-by-voxel R1-maps, generated from inversion recovery images using multiple inversion times. R1 is shown on a grayscale. Higher R1 values represent voxels where due to higher percentage of infarcted tissue a greater extent of contrast agent accumulates.

FIG. 2B illustrates the corresponding percent infarct map (PIM). The higher the PI, the more extensive the irreversible injury. Note that voxels with close to 100% infarct were mainly found sub-endocardially, while patchy infarct was mostly sub-epicardial and at the infarct borders, where salvage is most likely upon reperfusion.

FIG. 3A illustrates an equatorial short axis voxel-by-voxel PIM. Percent-Infarct is shown with a range of 0-100% of infarcted tissue per-voxel.

FIG. 3B illustrates the corresponding TTC-stained photo, which confirmed that PIM localized the infarct accurately. Although TTC-staining only shows the surface of a slice, it confirmed the tortuous morphology of the infarct and the residual viability subepicardially in the regions marked with arrows. In these regions infarcted and viable tissue are mixed. PIM visualized the patchiness and quantified each voxel individually.

FIG. 4A illustrates multislice short-axis and three-chamber long-axis PIMs 24 h after PCA administration in a dog with anteroseptal infarction.

FIGS. 4B-4D illustrate a 3D reconstruction of the left ventricle (LV) viewed from the apex (FIG. 4B), from the base (FIG. 4C) and from the septum (FIG. 4D).

FIG. 4E illustrates the long-axis view.

FIG. 5 illustrates the pair-wise correlations in six dogs between PIS determined using PIM₂₄ₕ (open squares) or PIM₄₈ₕ (filled diamonds), both versus TTC-staining. Regression lines (not shown) were y=0.9+1.5 (R=0.97, p<0.01), and y=0.83x+0.9 (R=0.97, p<0.01), for PIM₂₄ₕ and PIM₄₈ₕ, respectively. Line of identity is shown as a dotted line. PIS, determined from PIM at either time points, was not significantly different from TTC results (p<0.05).

FIG. 6A illustrates short axis multislice cardiac SI₀ maps in a dog 48 h after myocardial infarction. SI₀ is the time independent parameter, which contributes to the signal intensity and is mainly dependent on magnetic field strength and proton density. The proton density in the left ventricular chamber blood is higher than in the myocardium represented by the higher SI₀ values in the chambers. Note also the high inhomogeneity of SI₀ within the myocardium, partly due to coil-effect. This is the reason that regions closer to the coil (anterior regions, denoted by white arrows) display higher signal intensity (SI) values than regions more distant from the coil (posterior regions, denoted by black arrows).

FIG. 6B illustrates a voxel-by-voxel native T1 map obtained for the purpose of determining baseline R1 in the same dog. Myocardial T1 was on average 975+/−45 ms, which was in agreement with other investigator's results at the same field strength.

FIG. 6C is a parametric map of correlation coefficients for the voxel-by-voxel fitted relaxation curves. The average R² value in the myocardium was 0.99+/−0.005.

FIG. 7A illustrates short axis multislice delayed-enhancement (DE) images 20 minutes after the administration of 0.2 mmol/kg Gd(DTPA). Due to the nulling of viable myocardium, epi- and endocardial borders of the LV can not be delineated accurately.

FIG. 7B illustrates the thresholded (Remote+2SD) DE images (2-BIT BINARY IMAGES).

FIG. 7C illustrates a voxel-by-voxel PIM in the same dog. The higher the PI, the more necrotic, contrast-agent accumulating tissue there is in the voxel represented by the given pixel in the image. Note that PIM provides information about the patchiness of the infarct, and in each voxel it yields the percentage of infarcted vs. non-infarcted tissue. Thus, the 3D information hidden in the 2D MRI images is not sacrificed in the analysis, but on the contrary, it is utilized to obtain information about the density and distribution of infarcted tissue.

FIGS. 8A through 8F illustrate equatorial short-axis images of the left ventricle generated in a test dog 48 hours after the reperfusion of a myocardial infarction. FIG. 8A illustrates delayed enhancement (DE) image acquired 15 minutes after administration of Gd(DTPA). Signal in viable, remote myocardium is nulled, while infarcted regions appear enhanced. Note that the signal within the infarcted region is not homogeneous, probably due to different percentage of infarcted vs. non-infarcted cells present. Inhomogeneity of signal is greater in the subepicardial regions, where salvage is most likely to occur on reperfusion, and where viable tissue islets are most likely to be found.

FIG. 8B illustrates a thresholded DE image generated from the image shown in FIG. 8A. Note that all voxels, represented by enhanced pixels, are considered 100% infarcted with this method. Thus, in some voxels, where infarcted and viable tissue is mixed, viable tissue remains undetected. This may be one reason for DE overestimating infarct size.

FIG. 8C illustrates a voxel-by-voxel R1 map generated 20 minutes after Gd(DTPA) administration. The greater the contrast agent concentration in the tissue volume represented by the voxel, the higher the R1 value in a voxel. The extent of contrast agent accumulation depends on the amount of infarcted tissue in the voxel. Thus, R1 is proportional to the amount of infarcted tissue per voxel.

FIG. 8D illustrates a PIM, calculated voxel-by-voxel from the image in FIG. 8C. Note the higher percentage of infarcted cells in subendocardial infarcted regions, and the decreasing percentage towards the epicardium and infarct borders, where salvage is most likely to occur. The tortuous shape of the infarct can be visualized, because this method analyzes the content of each voxel (in depth), thus, in effect, provides sub-voxel resolution viability images.

FIG. 8E illustrates a corresponding TTC-staining photo showing the tortuous shape of the infarct. Note that this technique is only capable of showing the surface of the myocardial slice, thus, in theory, TTC is inferior to PIM, because it is based on the assumption that all cells below the surface of the slice are just like the one displayed on the surface. In reality, this is not so. While infarcted tissue is confluent in subendocardial regions, it appears patchy in subepicardial regions. Brownish-purple regions in the center of the infarct are hemorrhagic.

FIG. 8F illustrates a processed TTC photo for measuring infarct size, and infarct percentage per slice of left ventricle. Note the similar epicardial invaginations of viable areas shown by both the images shown in FIGS. 8D and 8F, but missing in FIG. 8B.

FIG. 9 illustrates a graph of Percent Infarct per Slice (PIS_{PIM}) versus Infarction Fraction (IF_{PIM}).

FIG. 10 illustrates a graph of the Percent Infarct per Sector (PISC) values of sectors where PISC>10% from PIM with their corresponding EDWT 8 weeks later.

FIG. 11 shows a correlation and linear regression analyses for resting ESWT at 8 weeks vs. the early PISC_{PIM} (R=−0.75).

FIG. 12 illustrates a simulated magnitude SI values in 100% infarcted (open squares) and in remote, viable myocardium (open diamonds) at different TIs. Contrast was calculated by subtracting the viable SI from the infarct SI (filled triangles).

FIG. 13 illustrates the correlation coefficients obtained for comparison of SI images with R1 images as a function of TI.

FIG. 14 illustrates the correlation between normalized SI-images and parametric R1-images. R1 and SI₀ values were obtained from the three-parameter curve fitting of the 10-TI data.

FIG. 15 illustrates two examples of equatorial short axis SI₀ (top) and A′ (bottom) maps. The ones on the left were generated in a test dog before contrast agent (CA) administration and those on the right were generated in the same dog 20 minutes after Gd(DTPA) administration. Average (+/−SD) SI₀ values were 142+/−22 and 150+/−23 before and after CA administration, respectively. Note the great variability of SI₀ values due to field inhomogeneity and regional differences in proton density. Average (+/−SD) A′ values were 1.9+/−0.01 and 1.9+/−0.02 before and after CA. No significant change due to the administration of Gd(DTPA) in either SI₀ or A′ were detected.

FIG. 16 illustrates two examples of equatorial short axis T1 maps. The one on the left was generated from multiple IR images acquired with six different TIs between 15-20 minutes after contrast agent administration. The one on the right, however, was generated from the SI₀ map, and only two of the IR images (TI=600 ms and 800 ms). Thus, it has been confirmed that once the SI₀ map is obtained, as few as two TIs are sufficient for an accurate T1 determination.

FIG. 17A illustrates images of an equatorial short-axis slice (top row) acquired with varying TE ranging from 11.2 to 106 ms for T2 mapping. Images were generated at 96 h following reperfusion of a 180-minute occlusion of LAD.

FIG. 17B illustrates a voxel-by-voxel color-coded T2 map showing increased T2 values in the injured region (arrowheads) due to increased water content. T2 averages were: T2 remote=5 3+/−2.5 ms; T2 infarct=67.7+/−6.6 ms.

FIG. 17C illustrates an SI₀ map of the myocardium showing changes in field strength and proton density from voxel-to-voxel. SI₀ values ranged from 390 to 1070, with an average of 695+/−185. Anterior regions displayed higher SI₀ values (white arrow) than those further away, in the posterior region (black arrow). This was partly due to the proximity of the coil (field inhomogeneity) and partly due to increased proton density in injured regions.

FIG. 17D illustrates a correlation-coefficient map of the myocardium that confirmed the excellent quality of curve fitting applied to the individual voxels' SI vs. TE dependence. The average was: R2=0.985+/−0.01.

FIG. 18 illustrates a graph of average R2 values in the infarcted regions (as delineated by Delayed Enhancement). Infarct R2 was significantly different from remote R2 throughout the first week. Lowest infarct R2 was detected on day 6 (11.8 s−1), which represents the peak edema. Edema retreated and R2 retuned to baseline by day 14. The highest R2 was detected in mature scar at 8 weeks (27.45 s⁻¹). Remote R2 stayed constant, and was not different from the baseline (gray horizontal line) (18.7 s−1) at any of the time points over the course of 8 weeks.

FIG. 19 illustrates equatorial short axis Percent-Edema-Maps (PEMs) are shown on days 3, 6, 14 and 56 following myocardial infarction in a test dog that was followed for 8 weeks. The percent-edema (PE) values are shown on a color scale showing varying extent of edema and scar maturation. The corresponding TTC-stained heart slice is shown on the right. The presence of myocardial edema was clearly detectable throughout the first week (black arrows). On day 3, edema is clearly apparent in and around the infarcted region. Maximum edema was detected on day 6, and it was almost completely resolved by day 14. The newly formed scar tissue, shown by gray arrows, had a PE value close to that of the normal myocardium. In the course of maturation, however, the scar tissue shrank and lost water, resulting in negative PE values by day 56 (white arrows). Note also the wall thinning over the course of the 8-weeks in these regions.

FIGS. 20A through 20I illustrates equatorial short-axis MRI images and post-processed parametric maps are shown in the same dog as in FIG. 19 on day 6 following a non-hemorrhagic infarction. FIG. 20A illustrates T2-weighted (T2w) image. Increased signal in the septum is due to the closeness of the coil (white arrows).

FIG. 20B illustrates a thresholded T2w image. Gray pixels are classified unaffected, white pixels are classified as edematous. Black arrows point to a region that does not appear enhanced in a T2w image (far from the coil), while it is in fact edematous (increased T2 values are obvious on the T2 map in FIG. 20D). White arrows point out the part of the septum that appears edematous (close to the coil), but in fact, it is unaffected (T2 values are normal on the T2 map in FIG. 20D and the region is far from the injured region).

FIG. 20C illustrates a parametric SI₀ map. SI₀ is mostly influenced by proton density and field inhomogeneity.

FIG. 20D illustrates a parametric T2 map. T2 is an intrinsic parameter of the tissue not influenced by field inhomogeneity or proton density. In edematous regions, T2 is elevated due to increased water content.

FIG. 20E illustrates a parametric R2 map calculated from the T2 map.

FIG. 20F illustrates a percent-Edema-Map (PEM) calculated from the R2 map. Gray arrows bracket the edematous (injured) tissue.

FIG. 20G illustrates a delayed-enhancement (DE) image. Infarcted regions appear enhanced.

FIG. 20H illustrates a thresholded DE image. White voxels are irreversibly injured, necrotic.

FIG. 20I illustrates a Tissue-Characterization-Map (TCM) generated from FIG. 20F and FIG. 20H. Note that the edematous region surrounds the necrotic tissue, with greater extent of edema near the irreversible injury, and smaller extent of edema farther away.

FIGS. 21A through 21H illustrate a combination of tagged cine MRI and TTC staining. All images were re-scaled to have a resolution of 10 pixels/mm. The end-diastolic (ED) voxel-by-voxel PIM is divided into sectors by the tagging grids of the corresponding ED tagged cine image. The PI values of all voxels in these sectors are summed and results are compared pairwise to results from the TTC slice analysis (Tag-sector-Percent-Infarct values). The end-systolic (ES) TTC slices are divided to sectors by transferring the ES tag-grid of the tagged cine image to the modified TTC photo. The number of infarcted vs. viable pixels in each TTC sector is expressed as a percentage of the total myocardial area in that sector. Results for corresponding sectors are compared pair-wise. FIG. 21A is an ED FIESTA. FIG. 21B is an ES FIESTA. FIG. 21C is an ED TAGGED CINE. FIG. 21D is an ES TAGGED CINE. FIG. 21E is a PIM (ED) 48 h after PCA. FIG. 21F is a TTC (ES). FIG. 21G is a ED tag-grid superimposed on PIM for sectoring. FIG. 21H is a ES tag-grid superimposed on TTC for sectoring.

FIGS. 22A through 22J illustrate equatorial short-axis MRI images and post-processed parametric maps are shown in the same dog as in FIG. 21, 4 days following hemorrhagic infarction.

FIG. 22A illustrates a delayed-enhancement (DE) image.

FIG. 22B illustrates a thresholded DE image. White voxels are irreversibly injured, necrotic.

FIG. 22C illustrates a T2-weighted (T2w) image. Increased signal in the septum is due mainly to the closeness of the coil (white arrows).

FIG. 22D illustrates a thresholded T2w image. Gray pixels are classified intact, while white pixels are classified as edematous (black arrows). Note that the crude method of thresholding T2w images not only overestimates the edematous region but is also unable to differentiate regions with varying extent of edema, and, therefore, T2w imaging cannot differentiate hemorrhagic from non-hemorrhagic infarcts.

FIG. 22E illustrates a parametric R2 map calculated from the T2 map. R2 is an intrinsic parameter of the tissue not influenced by field inhomogeneity or proton density. In edematous regions, R2 is decreased due to increased water content.

FIG. 22F illustrates a Percent-Edema-Map (PEM) calculated from the R2 map. The region at risk is shown by black arrows. Note the apparent lack of edema in the center of the infarct due to acute hemorrhage in the infarct.

FIG. 22G illustrates a Tissue-Characterization-Map (TCM) generated from FIGS. 22F and 22B. Note that the edematous region surrounds the non-hemorrhagic part of the necrotic tissue, and the hemorrhagic region is in the center of the infarcted region (mainly subendocardially).

FIG. 22H illustrates a corresponding TTC-stained slice. Purple-brown region in the center of the infarct is hemorrhagic.

FIG. 22I illustrates a post-processed TTC-photo, where the hemorrhagic region can be delineated clearly as a light brown region within the infarcted region (gray arrowheads).

FIG. 22J illustrates a red channel of the original TTC photo, showing infarct borders most accurately.

FIGS. 23A through 25D illustrate short-axis slices of the left ventricle that are shown at 8 weeks after the creation of a reperfused myocardial infarction in an additional test dog. FIG. 23A illustrates a Delayed Enhancement Image. Gd(DTPA) accumulation and consequent hyperenhancement is apparent in chronic scar (bracketed by gray arrows).

FIG. 23B illustrates a Percent-Edema-Map (PEM) highlighting mature scar due to decreased water content (“negative edema”). Depending on the maturity of the scar, water content varies as shown coded by the varying hue of the purple end of the color scale.

FIG. 23C illustrates a Tissue Characterization Map (TCM). Practically the entire infarct (as detected by DE) is identified by TCM as chronic scar, and there is no significant edema surrounding the infarcted region either. Thus, this infarct cannot be mistaken for an acute infarct.

FIG. 23D illustrates the corresponding TTC stained photo showing mature collagenous scar.

FIG. 24 illustrates a size of the hemorrhage per-slice as determined in vivo using MRI Tissue Characterization Maps (TCM) in 3 dogs versus the infarct size per-slice as determined with TTC-staining. Each dog is denoted with a different symbol.

FIG. 25 illustrates a correlation between Percent-Hemorrhage-per-Slice (PHS) determined by TCM and PHS determined using TTC-staining.

FIG. 26 illustrates a processing step of TTC-stained photographs. The final result is a TTC-Tissue-Characterization-Map, which shows viable myocardium (blue), non-hemorrhagic infarct (yellow), and hemorrhagic infarct (red). To validate the macroscopic TTC findings 5 μm thick H&E stained microscopic slides of typical regions were generated where all three tissue types (viable tissue, non-hemorrhagic-infarct, hemorrhagic infarct) could be found (an example is shown in the left upper corner of the Figure). Excellent agreement between macroscopic and microscopic histology was found. In non-hemorrhagic necrotic regions, observed were the classical signs of karyolysis, loss of cross-striations, appearance of contraction bands, polymorphonuclear (PMN) leukocyte infiltration, and interstitial edema. In hemorrhagic infarct regions, however, extravasated red blood cells were prominent among the cardiomyocytes, along with karyolysis, contraction bands, and interstitial edema. The PMN infiltration is less prominent in these regions, due to the destruction of microvasculature (hence the hemorrhage) and consequent limited access to recruited PMN cells. For the same reason, removal of necrotic tissue commences at the periphery of the infarct.

FIG. 27 illustrates a calculation of percent-infarct (PI) values. R1 is the relaxation rate measured in presence of an infarct-avid CA. R1,0 is measured in absence of the CA. ΔR1 is the relaxation rate enhancement induced by the CA. Examples for remote (A), partially-infarcted (B), and completely-infarcted (C) myocardial voxels are shown. ΔR1ᵣ is the baseline relaxation rate enhancement in all myocardial voxels due to the systemic administration of CA. ΔR1_{c} is the relaxation rate enhancement, in addition to ΔR1ᵣ due to the infarct specificity and accumulation of the CA in 100% infarcted tissue (infarct core). ΔR1ᵥ is the relaxation rate enhancement in a voxel from a patchy infarct region. ΔR1ᵥ can range from zero to ΔR1_{c}. This dynamic range is the basis for the percent-infarct calculation. Bottom panel: voxel-by-voxel equatorial PIM with PI indicated on a heat-color scale.

FIG. 28 illustrates a graph that shows the correlation between tissue water content and myocardial transverse relaxation rate (R2) The theoretical R2 (0.27 s−1) for zero dry to wet weight ratio (DWR=0) represents 100% water content (as measured in distilled water). This value is used as the theoretical upper limit of edema (percent-edema (PE)=100 (open diamond). The average (n=5) in vivo healthy control myocardial R2 of 18.7±1.2 s⁻¹ and the corresponding DWR of 0.23±0.01 were determined in our recent experiments, and taken as a reference for PE=0 (filled diamond). These two well-defined points determine a straight line, which can be used to convert any observed myocardial R2 value into water content expressed as PE. In these experiments the lowest R2 detected (peak edema on day 6) was 7.4 s⁻¹, which corresponds to a DWR of 0.083 (gray circle). Based on the straight line this converts to a PE of 61. The highest R2 detected, in collagenous scar (on day 56), was 28.6 s⁻¹, corresponding to a DWR of 0.35, and converting to a PE of −54 (gray square). Thus, the dynamic range of R2 values extends from 7.4 to 28.6 s⁻¹ with PE ranging from 61 to −54, demonstrating the robust changes in R2 due to changes in water content associated with post infarct tissue changes.

FIG. 29 illustrates the Tissue-Characterization-Mapping (TCM) algorithm. Based on the Percent-Edema-Map (PEM) and the corresponding delayed enhancement (DE) image, the TCM is generated using the color codes shown above.

### DETAILED DESCRIPTION

Methods of analyzing non-clinical parameters, methods of analyzing clinical parameters, methods of producing visual images, systems of analyzing non-clinical parameters, systems of analyzing clinical parameters, systems of producing visual images, systems of producing reports, reports based on imaging modality information, and displays based on imaging modality information, are disclosed.

Embodiments of the present disclosure relate to obtaining, generating, and/or measuring imaging modality information pertaining to one or more non-clinical or clinical parameters in a structure (e.g, biological materials (e.g., tissue), non-biological materials, and the like) or a portion of the structure and generating and/or converting that information into a format (e.g., a quantitative visual display, a quantitative report, or a quantitative media used to convey information) that is useful for an interested party (e.g., a clinician or other person of interest).

In general, the visual images of the clinical parameters can be generated from a parameter or data (e.g., intrinsic and/or non-intrinsic physical parameters or measured/reconstructed signal) obtained using imaging modality techniques such as, but not limited to, magnetic resonance imaging (MRI), SPECT, PET, ultrasound, X-ray, CAT, and the like. For example, the parameters (e.g., intrinsic and/or non-intrincis physical parameters) or imaging modality signals are based upon the imaging modality measurement technique, and thus different imaging modalities can use different parameters or various sources of signals (e.g., nuclear magnetic resonance, radioactivity, absorption, emission, or the like) to produce the image and/or reports.

Typically, the imaging modalities provide information for two-dimensional regions (tomographic imaging) that inherently contain information from a three-dimensional region with the result of partial volume averaging, due to the fact that signals are obtained from matter with nonzero volume. Thus, the two-dimensional points (pixels) of an image are representations of three-dimensional volume elements of the tissue (voxel). This is typically a disadvantage that leads to, for example, blurring at the borders of different tissue types and lower quality of edge definition for organs or pathologic changes and errors in quantification of clinical parameters.

In contrast, the methods and systems of the present disclosure exploit the advantages of partial volume averaging to analyze clinical parameters, produce visual images, and the like. By acknowledging the fact that the raw data from imaging modalities is derived from three-dimensional voxels, methods and systems of the present disclosure can be used to extract quantitative information from the raw imaging data.

The advantages of such an approach include, but are not limited to, the following: If partial volume averaging across the imaged slab is not a disadvantage, the slice thickness can be increased, which may lead to shorter imaging time (smaller number of slices are needed to cover a given region), and better quality of signal, since the magnitude of the signal (thus, the signal to noise ratio) can be increased by increasing voxel size. Also, by individually quantifying clinical parameters per voxel on a continuous scale, rather than setting thresholds and categorizing image regions into affected or unaffected, more accurate measurements of clinical parameters can be obtained. When using intrinsic physical parameters, for example, for these purposes (e.g. relaxation rates in MRI), diagnostic methods can be standardized more easily. This can improve the comparability and reproducibility of studies across various MRI equipment and other imaging modalities.

In particular, information from the imaging modality (e.g., directly or indirectly measured, generated, and/or determined intrinsic or non-intrinsic physical parameters) that directly or indirectly pertains to or relates to one or more parameters (e.g., clinical or non-clinical parameters) in the structure (e.g, tissue), can be obtained, analyzed, and/or presented to the interested party (e.g., clinician) in one or more formats. In an embodiment, the information may be obtained and/or processed to provide a visual display (e.g., in an electronic format (e.g., computer display), non-electronic format (e.g., a printed image), and/or or as a data set) related to the clinical parameter as it pertains to the tissue. The visual display can be configured to convey useful information regarding the tissue and clinical parameters to the clinician. In another embodiment, the format includes a report that conveys information (e.g., via words or numbers) that is useful to the interested party.

It should be noted that the methods and systems described in the present disclosure could be used with clinical and non-clinical parameters, while the structures could be biological materials (e.g., organs, tissues, tumors, cells, and the like) or non-biological materials (e.g., structures composed of, but not limited to, a polymer, metal, mineral, composite, other materials in which homogeneity, strength, and/or integrity characteristics may be important, or a combination thereof). For example, the non-biological material could be a complex structure such as, but not limited to, a tire, a support beam, or other structure in which homogeneity, strength, and/or integrity characteristics may be important.

In an effort to clearly describe embodiments of the present disclosure, much of the remaining text corresponds to a discussion directed towards biological materials. However, the application of the methods and systems described herein also apply to non-clinical parameters and non-biological materials.

In one embodiment the visual display includes a “map” of the tissue or portion thereof. The map can include but is not limited to, a “parametric map”, a “functional map” or a “pathology map”. The term “parametric map” refers to the visual display of a given value (parameter) calculated from the originally acquired imaging data. The term “functional map” refers to a parametric map where the parameter calculated and displayed reflects a functional characteristic of an organ or tissue. The term “pathology map” refers to a parametric map where the parameter calculated and displayed reflects a change in tissue morphology, histology, and/or function, that is indicative of a process and/or a state (e.g., pathologic and/or physiologic).

The maps can be based upon and/or produced from one or more intrinsic and/or non-intrinsic physical parameters, either directly or indirectly. The maps can use one or more intrinsic and/or non-intrinsic physical parameters to generate maps corresponding to or relating to one or more clinical parameters. The maps can be a combination of maps or other visual displays from one or more imaging modalities (e.g., MRI or other appropriate techniques).

In short, the map can display one or more intrinsic and/or non-intrinsic physical parameters and/or calculated clinical parameters, individually or in combination, with other clinical parameters. In addition, the map can be used in conjunction with other visual displays to provide information to the clinician. The visual displays described herein are only illustrative and are not inclusive of the many ways in which a user could process data to produce the visual display.

The maps or other visual displays of the tissue provide a convenient format to indicate the presence/absence, change over time, and/or distribution of the clinical parameter in the tissue. The map or other visual display of the tissue can be used to aid clinicians in localizing and determining the severity of a given clinical parameter and in planning the appropriate therapy to treat the clinical parameter in the tissue.

As mentioned above, embodiments of the present disclosure provide accurate, noninvasive imaging modality methods and systems (e.g., MRI-based methods and systems) to analyze and/or distinguish a clinical parameter in tissue or a portion of a tissue (or a pertinent non-clinical parameter in a structure to be analyzed for any purpose). The clinical parameter can include parameters such as, but not limited to, a tissue pathology, a tissue response, as well as additional clinical parameters amenable to the imaging modality (e.g., MRI analysis), that are useful for decision making in a variety of clinical settings. Such determinations can be useful for processes that are physiologic and/or processes that are pathologic. In particular, the determination could be useful for, among other things, diagnosing a particular condition, evaluating treatment options for the condition, and planning effective therapeutic regimens for the condition, and assessing the efficacy of the therapeutic regimens.

Reference to “tissue” can include, but is not limited to, a tissue, portions of a tissue, an organ, and the like, in two- or three-dimensions. A wide range of tissue types can be studied using the methods and systems of the present disclosure and these include, but are not limited to, myocardial tissues, nervous tissue, lymphoid tissue, skeletal and smooth muscle tissue, bones and cartilages, tissues of various organs (e.g., the kidney, the liver, the spleen, the prostate, the uterus, the testicles, and the ovaries), and select portions of each. The present disclosure provides examples and discussion of viability and other studies on myocardial tissues as non-limiting examples of the present disclosure.

The conditions can include, but are not limited to, altered growth rate of tissues, cancerous transformation of tissues, inflammation or infection of a tissue, altered volume of a tissue, altered density of a tissue, altered blood flow in a tissue, altered physiological function, altered metabolism of a tissue, loss of tissue viability, presence of edema or fibrosis in a tissue, altered perfusion in tissue, and combinations thereof.

The clinical parameter is a parameter related to, directly or indirectly, the condition. The clinical parameter can include, but is not limited to, a tissue pathology parameter, a tissue response parameter, a physiological parameter, tissue viability, tissue edema, tissue metabolism, or a combination thereof. In particular, the clinical parameter is tissue pathology or tissue response. It should be noted that when referring to a condition, one or more parameters related to the condition could be obtained. Likewise, when referring to a parameter, a condition corresponds to the parameter. Throughout the disclosure reference is typically only made to either the condition or the parameter, but it should be understood that when one of the condition or parameter is mentioned, the other is understood to be referenced.

Tissue response can include, but not limited to, a response of the tissue to a therapeutic intervention or the progression of tissue pathology in a given tissue over time.

In particular, the clinical parameter may be related to a clinical condition. The clinical condition may be acute or non-acute. Acute events include, but are not limited to, heart attacks, strokes, other ischemic events, trauma, burns and conditions related thereto that may require immediate intervention. Non-acute (chronic or intermittent/episodic) conditions include, but are not limited to, diabetes mellitus, ischemic heart disease, neoplastic proliferation of tissues, other hyper-proliferative or disregulated cell/tissue growth, degenerative diseases of various tissues (e.g. multiple sclerosis of nervous tissue), autoimmune diseases, deficiency or overproduction of various enzymes, hormones or other mediators, chronic wounds, and similar injuries as well as commonly encountered disease states. A disease state is defined as a medically defined condition, which is subject to diagnosis by trained medical personnel. It should be noted that the definition of a disease state could change in the future, but such a change should not alter the application of the present disclosure.

In an alternate embodiment, the clinical parameter is the response of a tissue to therapeutic intervention directed to treat the clinical condition.

In yet another alternate embodiment, the clinical parameter is the progression of a clinical condition in a tissue.

For example, using tissue viability as a tissue pathology parameter, the ability to determine and differentiate viable myocardium from irreversibly injured myocardium is crucial for clinical decision making after episodes of cardiac ischemia. This would enable clinicians to identify patients that would benefit most from re-vascularization therapy. It is known that patient outcomes improve with a greater extent of myocardial viability. Therefore, it is important to determine the extent of myocardial viability for required therapy and evaluation of treatment results.

As mentioned above, the imaging modality can include, but is not limited to, MRI, SPECT, PET, ultrasound, X-ray, CAT, ultrasound and the like. The intrinsic physical parameters measured and/or obtained using the imaging modalities may be different among imaging modalities. Therefore, in an effort to clearly describe embodiments of the present disclosure, portions of the disclosure refer to the intrinsic physical parameters of MRI. However, intrinsic physical parameters of one or more of the other imaging modalities can be used in a manner consistent with the methods and systems described herein.

In one embodiment where the imaging modality is MRI, the methods and systems disclosed herein use, directly or indirectly, the longitudinal relaxation rate (R1), the irrecoverable transverse relaxation rate (R2) and the total transverse relaxation rate R2*, signal intensity (SI), signal intensity at equilibrium (SI₀), proton density (PD), or other MRI-derived parameters and combinations thereof, to generate a map of a tissue of interest. R1 is defined as 1/T1, while R2 is defined as 1/T2 and R2* is defines as 1/T2*. The methods and calculations of R1, R2, SI, SI₀, and PD are discussed in more detail in the Examples.

In an embodiment, the changes in R1, R2 or R2* values among tissue regions displaying different degrees of a clinical parameter are determined. From the differences in R1, R2, or R2* values of ΔR1, ΔR2 or ΔR2* parameters, respectively, can be determined. Raw data for these measurements can be obtained with a variety of signal acquisition methods such as, but not limited to, an inversion recovery type MRI acquisition sequence, spin echo acquisition sequence, gradient echo acquisition sequence, echo planar acquisition sequence and combinations thereof.

The use of ΔR1 and ΔR2 values to generate data to evaluate the clinical parameter liberates, if not totally at least in part, the acquisition of the data in any selected tissue, for example myocardium, from dependence on extraneous experimental factors (such as the coil effect) commonly encountered when using signal intensity values from MRI images acquired with various techniques (T1-weighted, T2-weighted, PD-weighted, diffusion-weighted, and the like). Therefore, the acquired ΔR1 and ΔR2 values more accurately reflect the clinical parameter of the tissue examined. In addition, the ΔR1 value detected in a given segment in any tomographic slice of tissue (i.e, of a given tissue volume element) under certain circumstances may represent the ΔR1 and ΔR2 values, obtained by, but not limited to, single or multi-exponential analysis, averaged over that volume element. Thus, a three-dimensional ΔR1 or ΔR2 map of the selected clinical parameter in a tissue, such as the heart, can be constructed.

Since the variations in ΔR1 and ΔR2 values are directly proportional to the variations in the clinical parameter analyzed, the ΔR1 and ΔR2 maps are faithful representations of the clinical parameter analyzed and with the same spatial resolution as of the R1 or R2 map itself. As mentioned above, the present disclosure provides methods (e.g., MRI methods) for the evaluation of a variety of clinical parameters based on R1 and/or R2 values obtained from tissues displaying different values of the clinical parameter. Using the ΔR1 and ΔR2 values, parametric coded maps as well as other maps can be generated to visualize and/or quantify information about the distribution of the clinical parameter in a tissue.

For example, if the clinical parameter to be analyzed, such as myocardial tissue viability, is related to a tissue pathology and an infarct-avid contrast agent is used, the ΔR1 map is a faithful representation of agent distribution within the myocardial tissue, and thus, of viability or lack of viability, in the tissue region of interest.

In one embodiment, the change in R1 or R2 values, or ΔR1 and ΔR2, respectively, among tissues displaying different degrees of a clinical parameter is large enough so that contrast agents are not required. In other embodiments, the change in R1 or R2 values, or ΔR1 and ΔR2 respectively, among tissues displaying different degrees of a clinical parameter cannot be accurately assessed without the aid of a contrast agent.

The contrast agent increases the difference in R1 or R2 values between tissues that possess different values of a given clinical parameter by differentially entering the two tissue types, either normal or abnormal. For example, when the clinical parameter is tissue viability, such as myocardial viability, the contrast agent used may preferentially accumulate in the non-viable myocardial tissue and alter the R1 or R2 in the non-viable myocardium such that a larger difference in R1 (and hence ΔR1) or R2 (and hence ΔR2) values between viable and non-viable myocardium can be obtained. In another embodiment, the contrast agent may accumulate in the viable myocardial tissue. In either instance, a larger difference in R1 or R2 can be obtained and/or measured.

In embodiments where contrast agents are used (e.g., ceMRI), the contrast-agent-induced alteration of R1 or R2, should not change significantly in the course of the execution of the R1 or R2 measurements. The time frame for such MRI acquisitions (i.e., the signal acquisition phase) varies with the number of tomographic slices necessary to cover the tissue of interest and with the imaging technique used.

In one embodiment where myocardial viability is to be determined, the acquisition time to obtain sufficient number of tomographic slices to cover the left ventricular (LV) myocardium is on the order of about 15 to 60 minutes. Therefore, the contrast agent used should have a sufficiently long-lived residence time in the tissue of interest, such as non-viable, infarcted myocardium, so that its concentration does not change during the signal acquisition phase to an extent that would cause a change in R1 or R2 measurement that is larger than the experimental error of the R1 or R2 measurement itself. Such a contrast agent is referred to as a persistent contrast agent (PCA).

In one embodiment, the PCA interacts with the non-viable infarcted tissue. In an alternate embodiment, the PCA interacts with the viable tissue. The PCA can include, but is not limited to, Gd(ABE-DTTA), and those described in U.S. Pat. Nos. 5,154,914, 5,242,681, 5,370,860, 5,804,164, which are each incorporated herein by reference. It should be noted that contrast agents having a sufficiently long-lived residence time in the tissue of interest thus that its concentration does not change during the signal acquisition phase to an extent that would cause a change in R1 or R2 measurement, can be used. The concentrations of the contrast agents used depend, at least in part, on the subject, the contrast agent, the tissue and the like. As such, the concentration can be selected and adjusted accordingly.

In one embodiment, one contrast agent of the family of Gd(ABE-DTTA) may be used as the PCA. As described herein, Gd(ABE-DTTA) specifically interacts with infarcted tissue, and has a rate of decay in the infarct within the acquisition time frame of about 30-40 minutes which is less than 1% of R1. Because of this slow change of R1, this decay does not impair the determination of R1 itself at any given time-point. Furthermore, Gd(ABE-DTTA) accumulates into the infarct area and it induces a considerable ΔR1, which is observable throughout the first week from administration.

Although Gd(ABE-DTTA) is referenced specifically and used in the examples below, the methods disclosed herein are not dependent on using any one particular PCA. Substances that meet the requirements as delineated above may be used.

It is contemplated that using fast imaging techniques may allow the use of contrast agents with fast tissue kinetics (such as Gd(DTPA)) as well to determine R1 and R2 accurately or to generate better tissue contrast. For accurate measurements, however, the above condition should be met, namely, that the acquisition time must be shorter than the time during which a significant change in contrast agent concentration occurs.

An additional advantage of using a PCA, such as Gd(ABE-DTTA), is that repeated ceMRI sessions can be carried out without the need for repeated administration of the PCA. Therefore multiple R1 or R2 measurements may be obtained over time and multiple maps or other visual displays for various clinical parameters generated over this time period. Such information would be of value to the clinician to assess the evolution of recovery and potential need for re-intervention. As discussed herein, Gd(ABE-DTTA) is still observable in the infarct region one week after administration.

It should also be noted that visual displays could be obtained and/or generated from signal intensity (SI) and/or proton density (PD). An advantage of using SI and/or PD individually or in combination with other parameters, is that SI and PD are additive parameters (i.e., each minimal volume element (MVE) has its contribution to the net value detected in a given voxel of interest). MVE can be defined as the largest volume within which the ensemble of protons (or another element examined) are diffusing, communicating, and exchanging nuclear magnetic states fast enough that for all practical purposes they appear to display one averaged value for each one of the pertinent physical parameters (PD, R1, R2, and R2*). Thus, for both SI and PD, each voxel yields a value that is the sum total of all the individual values in the MVEs constituting it.

In some embodiments, the generation of the visual display (e.g., percent pathology map) can be generated from signal intensity (SI) values for the entire organ of interest (e.g., heart) provided that a) a near-linear relationship can be shown between the tissue pathology and SI and/or local contrast agent concentration and b) that fluctuations of SI due to extraneous experimental factors (e.g., field inhomogeneity) are negligible in the context of the particular purpose.

The parametric map can include, but is not limited to, a percent pathology map (PPM), percent infarct map (PIM), percent edema map (PEM), percent perfusion map (PPM), tissue characterization map (TCM) virtual biopsy map (VBM), and combinations thereof. The VBM is a map showing the ratio of various types of tissues (e.g., neoplastic vs. normal) within the imaged region. The TCM is a combination of a delayed enhancement image and a PEM, which are described in more detail herein.

In an embodiment, the pathology map is based, at least in part, on an absolute percentage of a clinical parameter (or functions used to describe the clinical parameter) or the relative percentage of clinical parameter (or the relative percentages of functions used to describe the clinical parameter). It should be noted that additional details regarding the maps are discussed in the Examples.

In an embodiment, the percent pathology map (PPM) may be used to determine the pathology of tissue or portions of a tissue before and/or after an event. Appropriate parameters (e.g., R1, ΔR1, R2, ΔR2, PD, SI, SI₀, and the like) can be determined that reflect tissue pathology based on the different parameter values obtained from different portions of the tissue. In an embodiment, the tissue is enhanced with a contrast agent, while in another embodiment, the tissue is not enhanced with a contrast agent.

The percent viability map (PVM) may be used to determine the extent of viability of tissue. Using the R1 values obtained, measured, or generated, a ΔR1 can be determined that reflects tissue viability based on the different R1 values obtained from the tissue. In one embodiment, the percent infarct map (PIM) may be used to determine the extent of irreversible injury of tissue after an infarct event.

In an embodiment, the tissue is enhanced with a contrast agent, while in another embodiment, the tissue is not enhanced with a contrast agent. It should also be noted that other parameters such as R2 could be used to generate the PIM.

The percent edema map (PEM) may be used to determine the extent of edema of tissue. Using the R2 values measured ΔR2 can be determined that reflects water content (edema or loss of water) in the tissue based on the different R2 values obtained from different portions of the tissue. In an embodiment, the tissue is enhanced with a contrast agent, while in another embodiment, the tissue is not enhanced with a contrast agent. It should also be noted that other parameters such as R1 could be used to generate the PEM.

As mentioned above, the tissue characterization map (TCM) is a combination of a DE image and a PEM. The PEM is acquired in the same manner as described above, while the DE image is generated in a manner described in the examples. In an embodiment of the TCM, each region of the tissue can be identified and/or classified as a type of tissue. For example, the tissue can be identified and/or classified as one of the following: healthy tissue, edematous tissue, necrotic tissue, necrotic-hemorrhagic tissue, scar tissue, neoplastic tissue, and combinations thereof in various organs of an organism. Additional details regarding TCM are provided in the Example.

In a similar manner, for clinical parameter or tissue pathology, a PPM can be generated. The PPM can be created for a variety of tissue pathologies as described herein. When the tissue pathology is tissue viability, a PVM may be generated. In one embodiment of a PVM where the aim is the determination of the area of myocardial tissue viability following a myocardial infarct, the map may be referred to as a PIM, which displays areas of viable and non-viable (infarcted) myocardium. When the tissue pathology is the extent of edema, a PEM may be generated.

An additional embodiment of the present disclosure is the use of spatial tagging for achieving co-registration or confirming spatial correlation and co-registration of images among various types of MRI acquisitions or inter-modality comparisons such as, but not limited to, comparing end-diastolic (ED) MRI images or parametric maps to end-systolic (ES)-looking photographs of TTC-stained heart slices (FIG. 36). By generating multiple-phase tagged MRI-images of an organ, the movement and/or change in shape of particular tissue segments can be followed and recorded (e.g., the movement of myocardium during contraction and relaxation of the heart). By dividing the tissue of interest to sectors delineated by the tagging grid, the values within that region for a given parameter of interest can be determined. Since the tissue tagged “carries” the tags for a limited time, in spite of translation or distortion of the initial tissue segment, the same segment of tissue can be tracked and identified and delineated.

It should also be noted that the present disclosure includes systems and methods directed towards apparent relaxation rate-based determination of tissue clinical parameters (discussed in more detail in section L), increasing spatial resolution by frame-shifted R1 or R2 maps (as discussed in more detail in section M), and improved systems and methods of MRA (as discussed in more detail in section P).

The methods and systems of the present disclosure can be implemented in software (e.g., firmware), hardware, or a combination thereof. The methods and systems can be implemented in software, as an executable program, and is executed by a special or general purpose digital computer, such as a personal computer (PC; IBM-compatible, Apple-compatible, or otherwise), workstation, minicomputer, or mainframe computer, or the dedicated computer attached to, and part of, e.g., the MRI instrument.

The software in memory may include one or more separate programs, each of which comprises an ordered listing of executable instructions for implementing logical functions. The software in the memory includes the methods and systems in accordance with the present disclosure and a suitable operating system (O/S).

When the methods and systems are implemented in software it should be noted that the methods and systems could be stored on any computer readable medium for use by or in connection with any computer-related system or method.

In an alternative embodiment, where the methods and systems are implemented in hardware, the methods and systems can implemented with any or a combination of the following technologies, which are each well known in the art: a discrete logic circuit(s) having logic gates for implementing logic functions upon data signals, an application specific integrated circuit (ASIC) having appropriate combinational logic gates, a programmable gate array(s) (PGA), a field programmable gate array (FPGA), etc.

Now having described embodiments of the present disclosure in general, the following describes embodiments of the present disclosure in additional detail. The following discussion is not intended to be limiting to the embodiments, but rather provide additional details about some illustrative embodiments.

### EXAMPLES

The examples below provide illustration of the application of a number of embodiments of the present disclosure. The examples below are not intended to be limiting in nature, but to illustrate the principles described in the disclosure. The methods and systems disclosed could be used to determine nonclinical parameters, or other clinical parameters in other applications as well, including applications where contrast agents are not used and where the parameter manipulated to generate the relevant maps can include one or more of the parameters corresponding to the imaging modality.

### A) Percent Pathology Map (PPM)

As mentioned above, the percent pathology map (PPM) may be used to determine the pathology of tissue or portions of a tissue before and/or after an event. Appropriate parameters (e.g., R1, ΔR1, R2, ΔR2, or other imaging parameters obtained using other types of imaging methods.) can be determined that reflect tissue pathology based on the different parameter values obtained from different portions of the tissue.

As shown in the flow-chart, by knowing the baseline (normal) value for a given parameter in a given tissue and the maximum change caused by a physiological process, therapeutic intervention, and/or pathology, a quantitative scale can be determined to visualize and quantify the tissue distribution of the given parameter of interest.

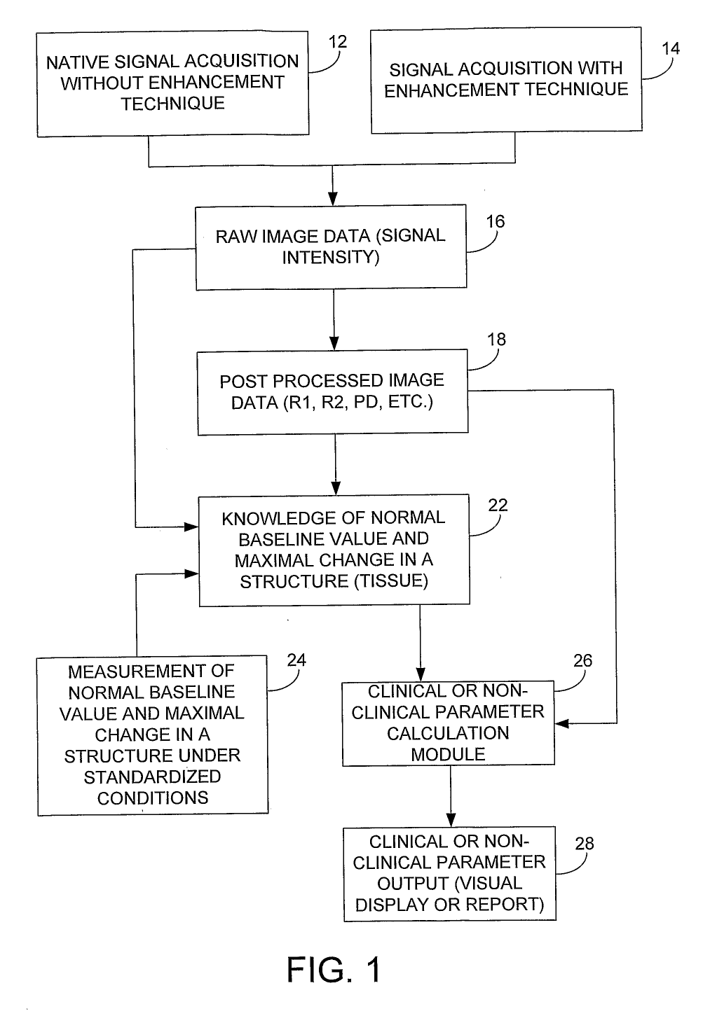

FIG. 1 is an illustrative flow chart describing an embodiment of generating a PPM map. In block 12, a native signal is acquired without enhancement for an imaging modality such as MRI. In block 14, a signal is acquired with enhancement. Both blocks 12 and 14 flow into block 16. In block 16, raw image data is obtained from blocks 12 and 14. Block 16 flows into both blocks 18 and 22. In block 18, the raw image data is post processed to provide post processed image data. Block 18 also flows into block 26. Block 24 flows into block 22. In block 24, measurements of a normal baseline and maximal change in a structure (e.g., tissue) under standardized conditions are provided. In block 22, knowledge and/or measurements of a normal baseline and maximal change in a structure (e.g., tissue) are provided. In block 26, the clinical or non-clinical parameter is calculated, while in block 28 the clinical or non-clinical parameter is used to produce a visual image and/or a report.

### B) Myocardial Viability Detection Using Percent Infarct Mapping (PIM)

The MRI methods and systems discussed above may be used to determine the extent of myocardial viability after a myocardial infarct. Using the R1 values acquired, ΔR1 can be determined that reflects tissue viability based on the different R1 values obtained from the viable and non-viable tissue.

R1 is composed of the R1₀ (the myocardial R1 in the absence of PCA or other contrast agent) and ΔR1 (the net paramagnetic contribution of the PCA or other contrast agent in the volume element examined). The ΔR1 values may then be used to generate a map or other visual display of the clinical parameter in a tissue of interest.

In one embodiment, a contrast agent is used to aid in the creation of a difference in the R1 value from viable and non-viable myocardium. Since the contrast agent distributes into all myocardial volume elements in proportion with the percent of infarcted, irreversibly damaged cells out of the total number of cells in the volume element, the three-dimensional map of ΔR1 can be transformed to reflect the percent infarct of a tissue and to generate a Percent Infarct Map (PIM) of the heart.

The methods to convert the ΔR1 data into percent infarct data and the PIM are described herein. A PIM assesses the infarct size, or the global parameter called “infarction fraction”, as well as the density (i.e., the percent of infarcted cells per myocardial volume) of the infarct with a resolution determined by the number of myocardial volume elements per total volume that the MRI device is able to provide.

Multi-slice PIMs could be used to make predictions of the extent and the time frame of myocardial recovery possible. Using a PIM generated as described herein, such prediction could be made even at a time when viability cannot be accurately assessed by contractile function because of post-ischemic stunning.

The ability to determine the density of infarcted/nonviable cells in a given volume element has many advantages. Areas termed “mixed” or “spotty” or “patchy” infarct areas have been observed by various methods that have been used to assess the extent of myocardial infarction. These areas reflect the fact that there are large volumes of injured areas that are not completely necrotic nor 100% devoid of injury, but rather are mixtures to varying degrees of infarcted and noninfarcted cells.

The ability to generate accurate PIMs to evaluate the density of viable myocardial tissue in a given volume element, therefore, would make a prediction of extent and time frame of recovery possible.

Typically, PIM is calculated based on R1. However, it should be noted that the calculation of PIM could also be based on T1-weighted SI, by taking the baseline SI of remote (PI=0%), viable regions and the maximum SI in the center of the infarct core (PI=100%).

In FIG. 2, multislice voxel-by-voxel R1 maps (A) and PIMs (B), generated from such images, are shown. The good quality and coregistration of images acquired with varying TI, was confirmed by the R² values obtained for the pixel-by-pixel curve-fitting applied to the SI vs. TI dependence. Average R² in the myocardium was 0.99±0.005.

Average R1₀ was 0.98±0.05 s⁻¹, in agreement with other investigators' findings. Average R1ᵣ, ΔR1ᵣ, R1_{c}, and ΔR1_{c} for both time points post PCA are shown in Table 1. Interpolating from the 24 h and 48 h R1 values, the hourly change in R1 in all myocardial areas between 24 and 48 hours post PCA was less than one percent of the measured R1 (0.0110.3 percent/hour for infarcted and 0.26±0.1 percent/hour for viable tissue), well below the experimental error of R1 measurements. Thus, it was confirmed that during generation of multislice R1 maps covering the LV (30-40 minutes), the concentration of Gd(ABE-DTTA) does not change to an extent that would significantly interfere with the accuracy of R1 measurement. This proves Gd(ABE-DTTA) to be a PCA suitable for multi-T1 R1 mapping.

TABLE 1
Average R1 and ΔR1 in remote regions(r) and infarct core(c)

| | 24 h after PCA | 48 h after PCA |
|---|---|---|
| R1ᵣ | 1.23 ± 0.06 | 1.15 ± 0.08 |
| R1_{c} | 2.33 ± 0.5 | 2.31 ± 0.4 |
| ΔR1ᵣ | 0.26 ± 0.03 | 0.18 ± 0.05 |
| ΔR1_{c} | 1.21 ± 0.5 | 1.27 ± 0.3 |

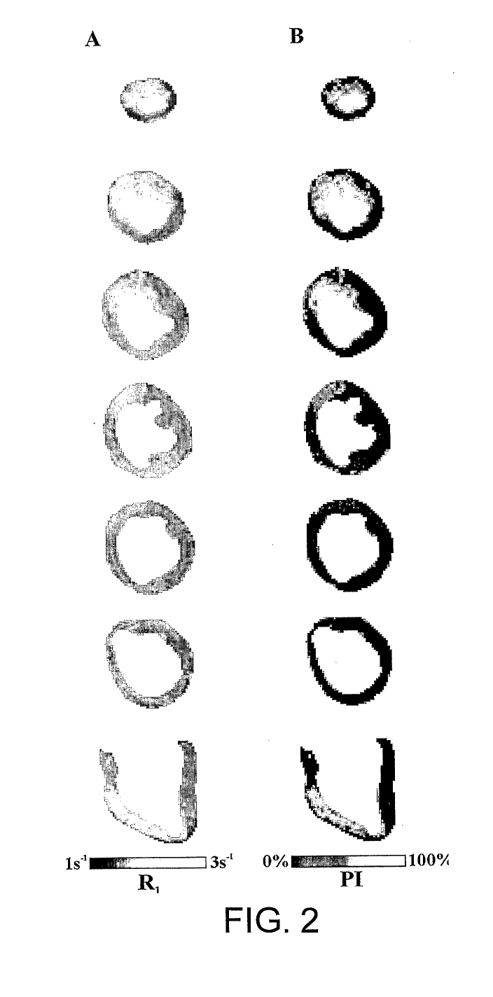

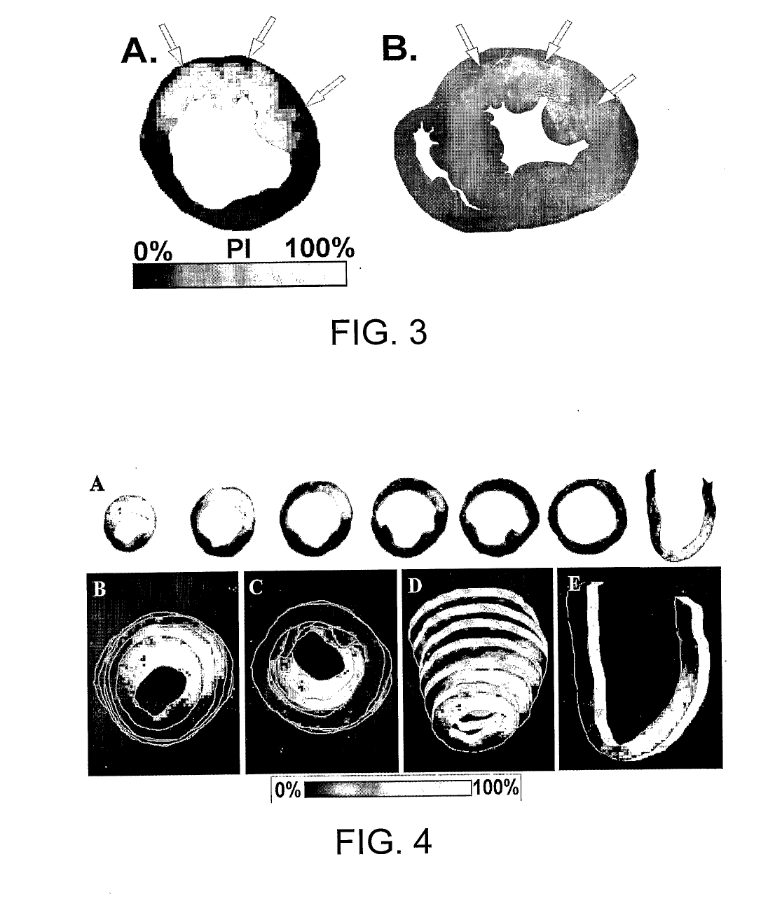

PIM localized the infarct to the exact same territory as TTC-staining. Equatorial short-axis PIM and a corresponding TTC slice are shown in FIG. 3 TTC-photos only show information on the “surface” of the slab, while PIM reflects data from the entire depth of the 10 mm thick MRI slab and gives information about the tortuous shape of the infarct border zone. PIM, in agreement with TTC-staining, detected partially viable (0<PI<100) regions along the infarct borders and in subepicardial, partially salvaged areas (indicated by arrows in FIG. 3). Solid infarct regions were found (high PI values) sub-endocardially.

FIG. 4 shows three-dimensional reconstructions of short-axis and long-axis PIMs covering the entire LV. The changing fraction of infarcted cells can be observed with high spatial resolution, and in a graded manner.

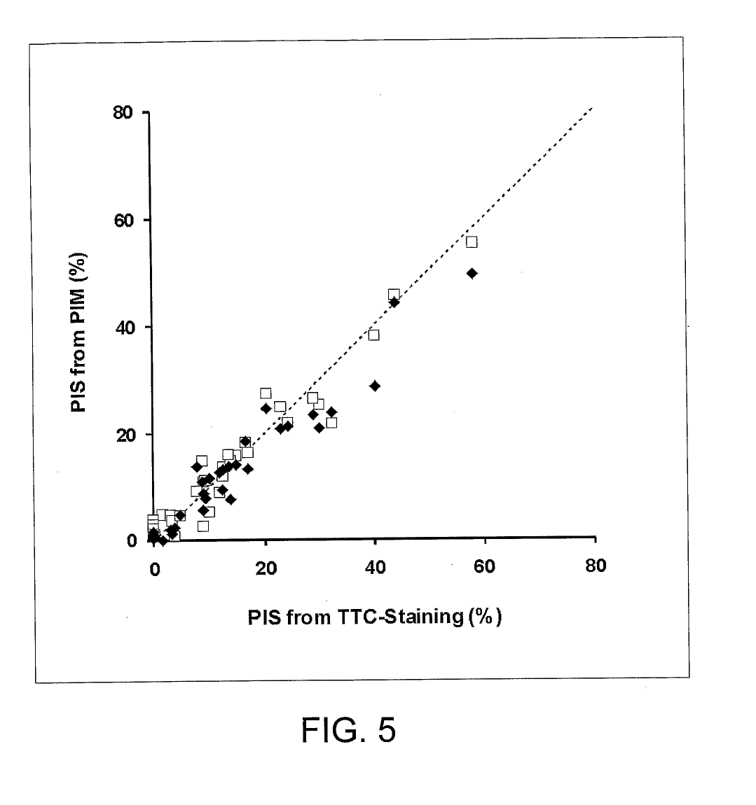

No significant difference appeared between TMM determined using PIM at 24 h (64±5 g) or PIM at 48 h (65±4 g), and neither was significantly different from TMM_{TTC} (65±5 g). Significant pairwise correlation was found for PIS_{PIM24h} (R=0.97, p<0.01) and for PIS_{PIM48h} (R=0.97, p<0.01), both vs. PIS_{TTC} (Table 2, FIG. 5). Also, strong significant correlation was obtained for IF_{PIM24h} (R=0.99, p<0.01) and IF_{PIM48h} (R=0.93, p<0.01) both vs. IF_{TTC} (Table 2). Average overestimations obtained with PIM₂₄ₕ and PIM₄₈ₕ as compared to TTC-staining are shown in Table 2. PIS_{PIM} and IF_{PIM} at both time points were not significantly different from those obtained with TTC-staining (p=NS).

TABLE 2
Average overestimations(±SD) of infarct sizes and correlation
coefficients (R) obtained for PIM vs. TTC-staining.

| | PIM | |
|---|---|---|
| | at 24 h | at 48 h |
| Overestimation of PIS_{TTC} (%) | 0.1 ± 3.5 | −1.5 ± 3.8 |
| Overestimation of IF_{TTC} (%) | 0.7 ± 1.1 | −1.0 ± 2.3 |
| Correlation coefficient for PIS_{PIM} vs. PIS_{TTC} | 0.97* | 0.97* |
| Correlation coefficient for IF_{PIM} vs. IF_{TTC} | 0.93* | 0.99* |

*p < 0.01

Excellent correlation was obtained for PIS_{PIM48h} vs. PIS_{PIM24h} (R=0.97, p<0.01). PIS_{PIM24h} and IF_{PIM24h} were slightly greater than those at 48 h, on average by 1.6±3.1 and 1.8±1.6%, respectively. These differences between the two time points for either PIS or IF, however, were not significant (p=NS) confirming the excellent reproducibility and stability of PIM.

### C) SI₀ Mapping

R1 values are typically calculated from the SI vs T1 dependence applying a three-parameter, bi-exponential least squares curve fitting routine using the equation:

SI=SI₀(1−A·e^{(-TI·R1)}+e^{(-TR·R1)})

where SI₀ is the signal intensity at equilibrium and A is a parameter dependent on the accuracy of the 180 degree inversion pulse and on the extent of saturation.

While SI₀ is obtained in the process of calculating R1 as described above, the actual value of SI₀ is generally not utilized. The value of SI₀ is governed to a large extent by proton density (PD), magnetic field inhomogeneity, and other confounders influencing the signal intensity at equilibrium. SI₀ practically may change from voxel-to-voxel throughout the imaged area and is a component of the detected SI value, independent of the imaging parameters (TI, TR) used, or of the intrinsic R1 parameter. SI₀ is also a reflection of the experimental conditions at the time of imaging, including effects of field inhomogeneity, the physical properties of the MRI equipment, coil position, patient position, and other confounders influencing the field.

By dividing the SI acquired with a given TI in each pixel by its corresponding SI₀ value, corrected SI maps (COSIM) are obtained. These COSIM maps (corrected MRI images) would reflect the intrinsic R1 parameters, and would detect more accurately tissue areas with varying, purely intrinsic parameters. Thus, MRI images (regardless of imaging parameters) could be “cleansed” from confounders that contribute to SI₀.

Once a baseline SI₀ map has been obtained, R1 could be estimated with great accuracy from images acquired with a fewer number of TIs (even from a single TI) and known TR. Thus, following the acquisition of a baseline SI₀ map (by generating a baseline T1 map), changes in the intrinsic R1 parameter due to therapeutic or diagnostic intervention (administration of a contrast agent) could be monitored. With the help of an SI₀ map, a series of IR images could be used to estimate R1. Especially when the acquisition of a complete set of TIs is not possible, due to time constraints, a smaller set of images acquired with one, two, or several TIs would be sufficient for R1 determination once the baseline SI₀ map is established.

A two-parameter non-linear curve fitting routine would be used for this purpose (once SI₀ is known) or the value can be determined directly. This method could be used for determining concentration maps of CAs with even fast-tissue-kinetics (such as Gd(DTPA)), as agent concentration is linearly related to ΔR1.

Following the acquisition of a baseline SI₀ map, the contrast agent would be administered and serial images using one or several TIs would be acquired. Using the method mentioned above could be used to produce serial R1 maps.

This could be used in acute ischemia to quantify tissue perfusion (to which CA concentration is proportional) from a single high-resolution image. Also, using this approach, the above mentioned PIM method could be employed even using CAs with fast tissue kinetics since fewer TIs, thus less time, would be required to generate the R1 or R2 values needed for evaluation of the clinical parameter.

The same principle can be applied for determining R2. Following the generation of a baseline T2 map and corresponding SI₀ map from images acquired with multiple echo times (TE), further T2 measurements in the same experiment could be carried out using fewer TEs and using the SI₀ map.

A further use of the SI₀ maps could be to help delineate organ contours. Endocardial borders of the heart may be difficult to define under certain circumstances, when the R1 values of the blood and the subendocardial myocardium (or endocardial infarct) are similar. The intrinsically higher PD in blood yields higher SI₀ values, thus blood and myocardium can be readily distinguished on an SI₀ map.

Similarly, due to differences in water content of blood and plaques, due to different water-PD, SI₀ maps may be useful in detecting vessel stenoses and atherosclerotic plaques.

### D) Percent Infarct Map Using Gd(DTPA), a Contrast Agent with Fast Tissue Kinetics and SI₀ Maps

A comparison between the correspondence of voxel-by-voxel percent infarct map (PIM) using Gd(DTPA) to TTC-based quantification of infarct and the correspondence of delayed enhancement (DE) to its TTC-based infarct quantification in vivo are now discussed.

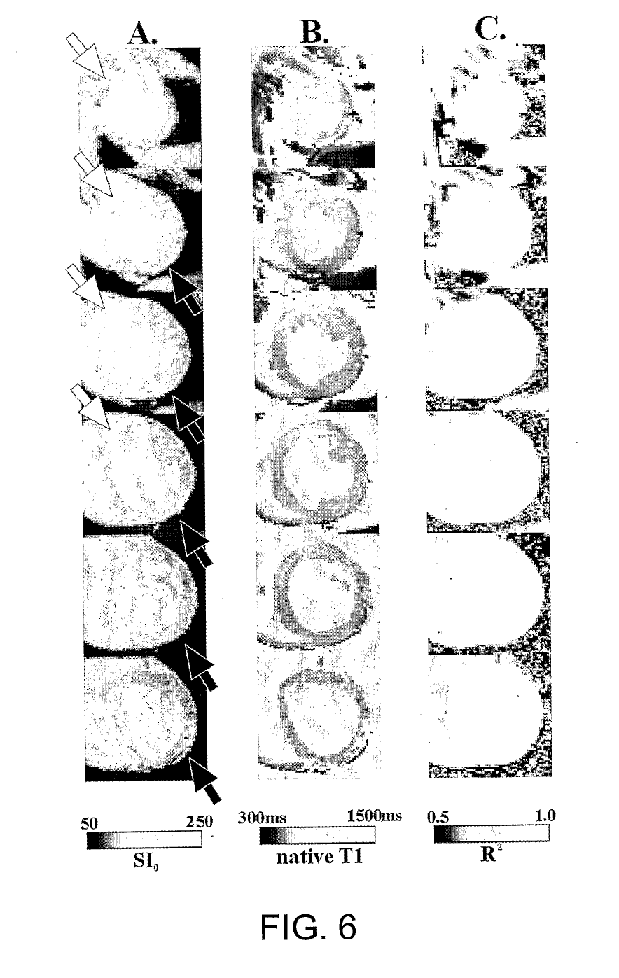

In three dogs (n=3), weighing 18-20 kg, myocardial infarctions (MI) were generated by a 180-minute closed-chest coronary artery occlusion. Forty-eight hours following reperfusion, baseline R1₀ map and SI₀-map (see below) were generated using the inversion-recovery (IR) technique with multiple inversion times (TI) (FIG. 6). Delayed-Enhancement (DE) imaging was carried out exactly 15 minutes after Gd(DTPA) (0.2 mmol/kg) administration.

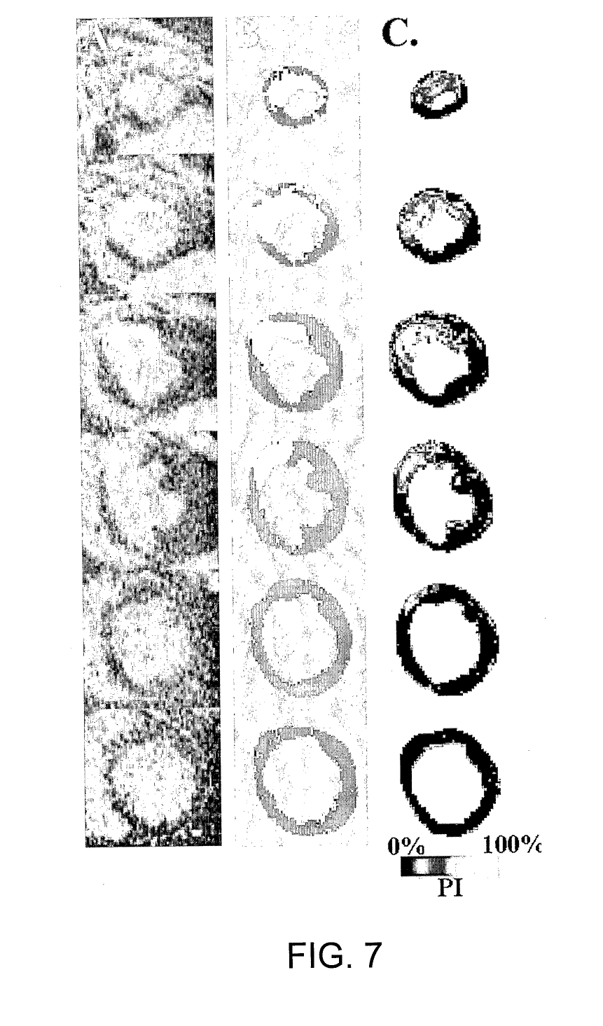

Both image acquisition and data analysis were carried out by scrupulously adhering to DE protocol as described in recent literature (Fieno D S, Hillenbrand H B, Rehwald W G, Harris K R, Decker R S, Parker M A, Klocke F J, Kim R J, Judd R M. Infarct resorption, compensatory hypertrophy, and differing patterns of ventricular remodeling following myocardial infarctions of varying size. Journal of the American College of Cardiology. 2004; 43:2124-2131), so that the results would not be biased. Endo-, and Epicardial contours were traced manually. Thresholding of DE images (Average Signal Intensity of remote myocardium+2SD) and infarct size determination in each MRI slice was automated to eliminate observer bias in delineating infarct area (FIG. 7B). Percent Infarcted area per Slice (PIS_{DE}) was calculated by counting enhanced pixels in the slice, and dividing them by the total number of pixels of LV myocardium in that slice. Infarction fraction (IF_{DE}) was determined by summing all infarcted pixels in all slices and expressing them as a percentage of the total LV myocardial pixel-count. After DE imaging, two IR images were generated with TIs of 600 and 800 ms for the purpose of R1 mapping (see below) and for generating PIMs (FIG. 7C).

The contrast-agent-enhanced SI values within the infarcted tissue areas were quite variable (FIG. 7A), as it had been also noted by other investigators. Such variability most likely results from the patchiness of the infarct, and the fact that infarcted tissue is mixed with viable tissue especially in the infarct rim, where salvage upon reperfusion is most likely to occur. Thus this variability of SI enhancement contains valuable quantitative information about the true distribution of infarct content. In the DE method, in the process of thresholding images, however, this information is lost. Pixels are categorized into two groups of tissue: either fully infarcted or fully noninfarcted. Thus, thresholding in fact reduces the high-resolution MRI image to a (YES/NO) binary image (FIG. 7B). PIM, however, offers insight into the composition of each voxel in depth (FIG. 7C).

Baseline parametric R1₀ maps, and corresponding SI₀ maps, that cover the left ventricle (LV), in a dog 48 h after myocardial infarction, are shown in FIG. 6. R1 was calculated from the SI vs. TI dependence as detailed herein. The calculated baseline relaxation rate of myocardium was R1₀=1.03±0.05 s⁻¹, which was later used for calculating the PIM.

Great inhomogeneity of SI₀ values, ranging from 50 to 230 SI units, was demonstrated in the myocardium. SI₀, according to the Equation describing the SI vs. TI dependence, is the time independent quantity.

SI=SI₀(1−A′·e^{(-TI·R1)}+e^{(-TR·R1))})

Thus, in case of local variations of SI₀, a wide range of SI values may be obtained throughout an image (acquired with a given A′, TI and TR) for exactly the same R1 values. This confirms that a single SI value by itself is an unreliable source of assessing contrast agent distribution.

SI₀ is determined mainly by local magnetic field strength and proton density. For instance, in FIG. 6 it was obvious that SI₀ in the LV chamber blood, in all slices, was greater than that in the myocardium in the same slice. This was clearly due to a difference in proton density. There was also great variation in SI₀ over the myocardium itself. While proton density can vary from voxel to voxel due to changing water content (inflammation, edema), field inhomogeneity also causes large differences among myocardial regions in a manner primarily dependent on their relative position to the receiver coil. Therefore, anterior regions appear brighter (white arrows), while posterior regions appear darker (dark arrows). There was also a gradient in the value of SI₀ from higher SI₀ values near the apex (top of FIG. 6) to lower SI₀ values near the base (bottom of FIG. 6). These findings confirm the unreliability of SI values acquired with a single TI, and provide yet another possible cause of error in infarct size assessment with the DE technique.

For the purpose of generating PIMs, 20 minutes after the administration of Gd(DTPA), high resolution images of the entire heart were acquired with two TIs using the same image localizations and sequence parameters (except the TI) as for the baseline R1₀ and SI₀ maps. Details of selecting the optimum TIs are discussed further below. From these images and from the corresponding SI₀ maps, voxel-by-voxel R1 maps were generated. Average R1 values in remote myocardium and infarct core were R1=1.73±0.07 s⁻¹, and R1=3.22±0.09 s⁻¹, respectively. R1 in LV chamber blood was R1=2.53±0.05 s⁻¹.

ΔR1 (ΔR1=R1−R1₀) was calculated automatically for each voxel utilizing the baseline R1₀ map. Average ΔR1ᵣ in the remote viable regions was 0.7±0.07 s⁻¹, while in the center of the infarct (denoted 100% infarcted) ΔR1_{c} was 2.19±0.09 s⁻¹. The contribution of any contrast-agent-accumulating infarct islet contributes to the total ΔR1 of that voxel. Therefore, ΔR1 is proportional to the amount of non-viable tissue present in any given voxel. Thus, a percent infarct (PI) scale was created, where the core ΔR1_{c} (maximum R1 enhancement observed in the center of the infarct) was considered 100% whereas the ΔR1ᵣ of remote areas was 0%.

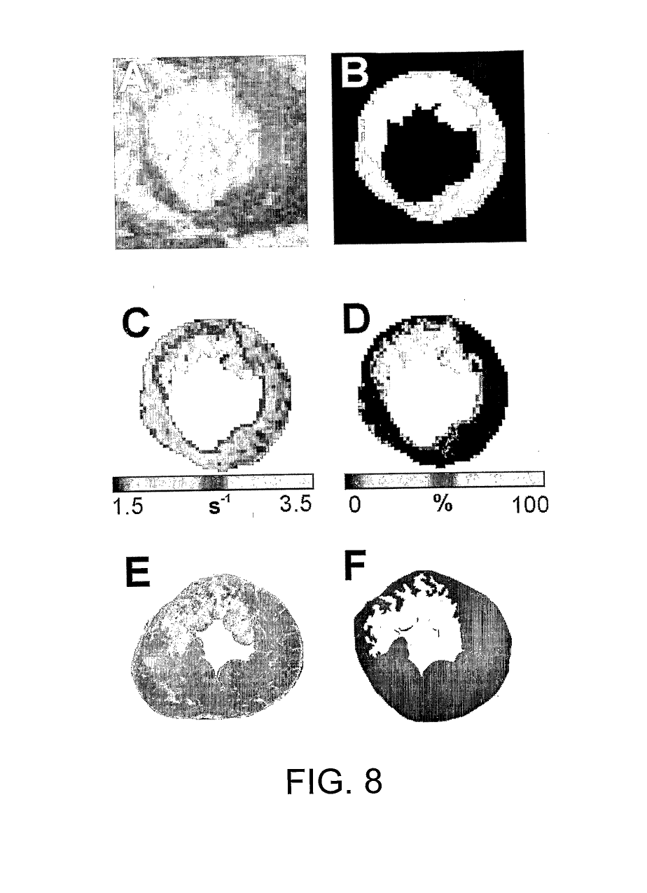

FIG. 8C shows an example of the voxel by voxel short-axis R1 map and FIG. 8D shows the PIM calculated from it. One advantage of the PIM method over thresholded DE images (FIGS. 8A and 8B) is that it utilizes the complex 3D information concealed in the 2D MRI images. The PIM method visualizes infarct morphology and distribution more effectively by quantifying infarct density per voxel, thus yielding a more realistic visualization of the tortuous morphology of infarcts. Since PIM is based on the intrinsic R1 parameter, another advantage of the PIM method is the elimination of extraneous experimental factors, such as field inhomogeneity, saturation, proton density, T2-effects, and the like. This, in turn, may also allow better standardization of results across different machines and different MRI sites.

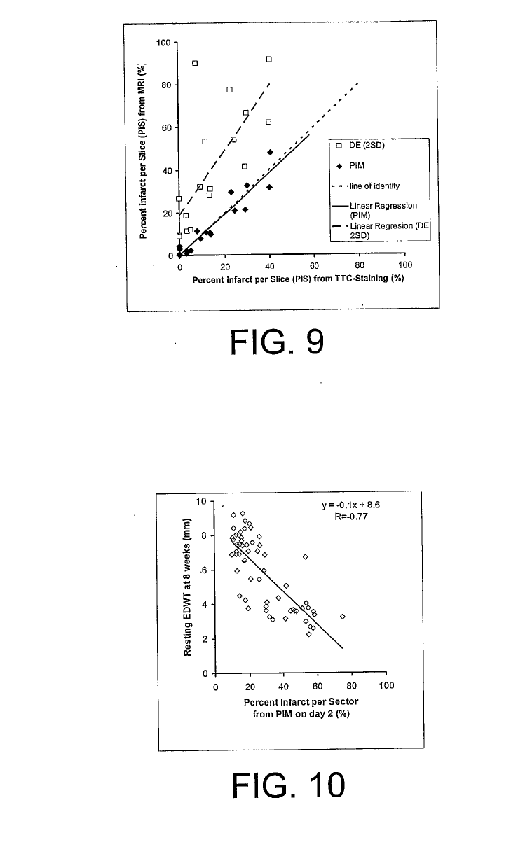

The advantages of PIM lead to a more accurate in vivo quantification of infarct size. In three dogs, Percent Infarct per Slice (PIS_{PIM}) and Infarction Fraction (IF_{PIM}) were calculated. Results from DE and PIM were compared head to head to the results from the gold standard, TTC-staining. Strong correlation was found between PIS_{PIM} and PIS_{TTC} (R=0.95) (FIG. 9). The regression line was y=0.96x+0.2. Average overestimation of infarct size per slice with PIM over the TTC-determined standard was a negligible −0.4±4.4%. DE (using an SIᵣₑₘₒₜₑ+2SD threshold), however, yielded a weaker correlation (R=0.74), and also systematically overestimated TTC-staining by an average of 26.1±20.3%. The regression line for DE(2SD) was y=1.51x+18.9. The results clearly demonstrate that PIM determines infarct size in vivo more accurately than DE.

### E) Predicting Long-Term Recovery Following Myocardial Infarction Using PIM:

In a separate group of three dogs (n=3), myocardial infarction were generated and carried out baseline R1₀-mapping, DE imaging and PIM generation 48 h following reperfusion in the same manner as in the dogs in the studies above in A).

Percent-Infarct-per-SeCtor (PISC) values were calculated from both the DE images and the voxel-by-voxel PIMs. Additionally, however, in this group SSFP cine MRI images were generated in the same image localizations as the DE and PIM images to record the decreased regional and global function due to the infarction. These dogs were then allowed to recover for 8 weeks to compare the predictive value of PIM with that of DE regarding function recovery.

Eight weeks following reperfusion, SSFP cine MRI images were generated in the same image localizations as on day 2. The coregistration of short-axis slices was ensured by a standardized technique of image angulation as well as taking into account anatomical landmarks such as the mitral and aortic valves. Cine images were analyzed as described herein, using a dedicated software dividing short axis images into 16 circumferential sectors. Since the PIM is generated with a voxel-by-voxel resolution, it offers great flexibility when results need to be compared with function or other imaging modalities. Voxel PI values can be integrated over any number of voxels with any type of division into sectors, as desired by the clinician or researcher.

End-diastolic, and end-systolic wall thickness (EDWT, ESWT) were measured on day 56 at rest, and during dobutamine stress. The day 56 function parameters were correlated with their corresponding early (day 2) PISC_{DE} and PISC_{PIM} values.

Note, that to prevent the large number of sectors little affected by infarction (i.e., with PISC<10%) from interfering with the correlation and regression analyses, these sectors were excluded from the function analyses and only analyzed sectors that with each method yielded a PISC value of >10%. Since DE overestimates infarct size, there were more sectors with PISC_{DE}>10% than there were with PISC_{PIM}>10%.

### F) Predicting Remodeling:

The parameter that best describes left ventricular (LV) anatomy, and thus LV remodeling, is EDWT. To demonstrate the ability of PIM to predict LV remodeling, first the PISC values of sectors were correlated where PISC>10% with their corresponding EDWT 8 weeks later (FIG. 10). Only very weak correlation was found for DE (R=−0.48, p<0.01), while a good correlation was found for PIM (R=−0.77, p<0.01). Thinner walls in sectors with high PISC values reflect scar shrinkage or formation of aneurysm. This suggests that the higher the PISC value early after an infarction, the thinner the sector 8 weeks later, demonstrating again the PIM method's predictive power.

The results clearly suggest that PIM is a more reliable predictor of LV remodeling than DE. The reasons for the PIM method's advantage over DE are similar to those mentioned above in the experiments to support A). While PIM is able to detect viable tissue in voxels that are only partially infarcted, DE counts all enhanced voxels 100% infarcted, regardless of the extent of enhancement. Thus, DE overestimates true infarct size per sector. This leads to the finding that remodeling (ΔEDWT<0, wall thinning) is not really observed in many sectors where PISC_{DE} is high.

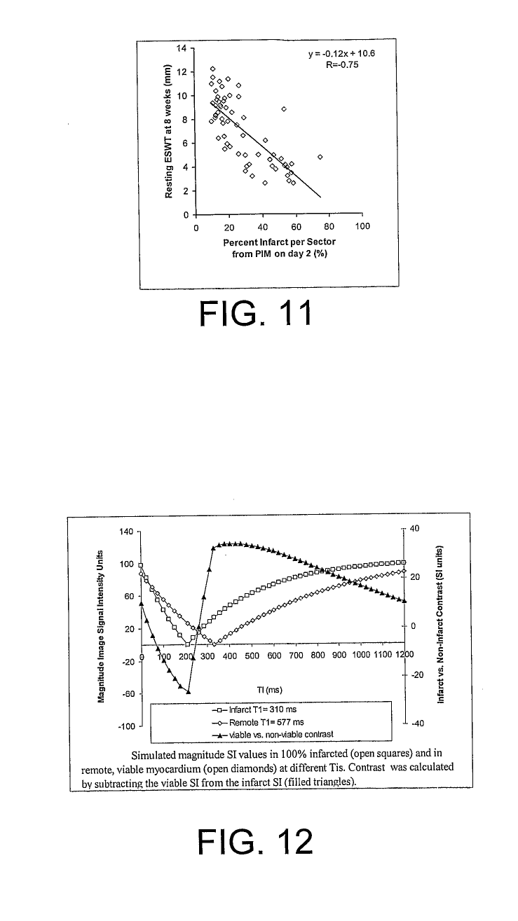

Predicting Recovery of Regional Function: The ultimate proof for tissue viability is the return of function to myocardial sectors that displayed impaired function due to an ischemic insult. To show the ability of PIM to determine viability after reperfusion and to predict future recovery of function, regional myocardial function parameters (ESWT) were correlated at eight weeks after reperfusion to the early PISC values obtained on day 2. FIG. 11 shows the correlation and linear regression analyses for resting ESWT at 8 weeks vs. the early PISC_{PIM} (R=−0.75).

Based on these results in three dogs it can be concluded that using the PIM method at an early time point after myocardial infarction, long term recovery of resting function and left ventricular remodeling can be predicted more reliably than with DE.

### G) Background for R1-Mapping Using the SI₀ Map

The simulation of the SI vs. TI dependence based on in vivo experimental results is discussed. Based on the in vivo measured T1 values magnitude SI values were simulated in 100% infarcted (open squares) and in remote, viable myocardium (open diamonds) at different TIs (FIG. 12). To find the maximum contrast with still a sufficiently high SNR in all regions, the difference between infarcted and non-infarcted SI was calculated (non-viable vs. viable contrast-filled triangles in FIG. 12), and plotted it in the same graph. This graph represents the dynamic range of SI values that voxels with infarct mixed with viable tissue would display.

Assuming a TR of 1200 ms, i.e., two R-R intervals at a heart-rate of 100/min, the τ₀ point of the remote regions is at 330 ms. Contrast at this point is 32.7 SI Units (SIU). Maximum contrast can be observed at 405 ms (34.6 SIU), but at this TI, SIᵣₑₘₒₜₑ is still low (13.4), resulting in a low SNR in IR images. At 600 ms, however, SIᵣₑₘₒₜₑ is up to 41.8 SIU, and the contrast is still high enough at 31.4 SIU (91% of maximum contrast).

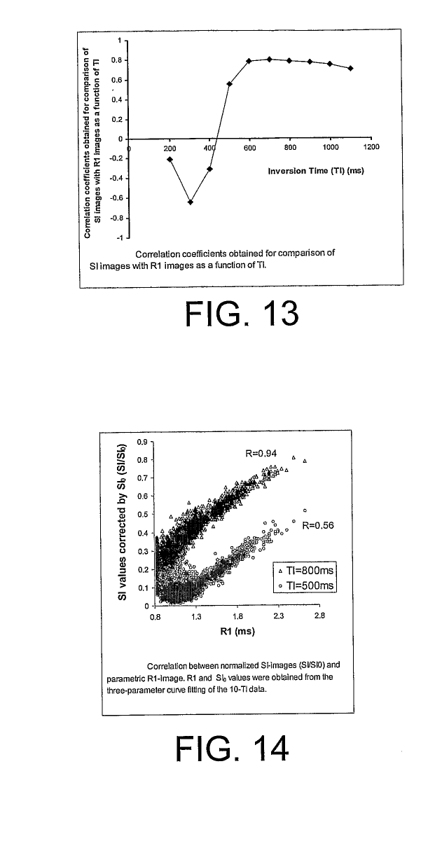

To find the optimum TI in vivo, R1 mapping was carried out in a test dog following the administration of an infarct avid persistent contrast agent, which causes a relaxation rate enhancement in infarcted tissue very similar to that observed with Gd(DTPA). This agent, due to its slow tissue kinetics, allowed us to generate IR images with ten different TIs. SI images (obtained with different TIs) were correlated with the R1 images (obtained from the three-parameter curve fitting of the 10-TI data) voxel-by-voxel. FIG. 13 shows the result of correlation analyses carried out for all 4000 myocardial pixels of this test dog. SI values obtained with TIs that were 100-300 ms longer than the to of remote myocardium (in this case TI=600-800 ms) yielded the strongest correlation with the intrinsic R1 values. Therefore these TIs yield the most reliable data for T1 mapping when time is of essence and only a few TIs can be used. Interestingly, at the τ₀ of remote myocardium (500 ms), the correlation was much weaker.

The same correlation analysis was conducted after dividing all SI values with their corresponding SI₀ values (normalizing for SI₀). This isolated extraneous confounders of SI from the correlation analyses. FIG. 14 shows the correlation plot of corrected SI values (SI/SI₀) obtained with TI=800 and TI=500 ms vs. R1. Correlation was much weaker with TI=500 ms (R=0.56) than with TI=600 ms (R=0.85, not shown in the graph for clarity) or with TI=800 ms (R=0.94). This means that the T1-weighted images, where signal in remote myocardium has to be nulled (e.g., DE), do not represent faithfully the intrinsic R1 parameter, which also means that they do not yield accurate information about the distribution of contrast agent administered. Another reason for favoring TIs longer than the τ₀ of remote myocardium is that in magnitude images the polarity of the signal is lost and for this type of calculation one has to make sure that all myocardial voxels have a positive SI value when calculating R1. These are at least some of the reasons for choosing IR images obtained with TIs of 600 and 800 ms for the purpose of calculating R1 with the help of the SI₀ map.

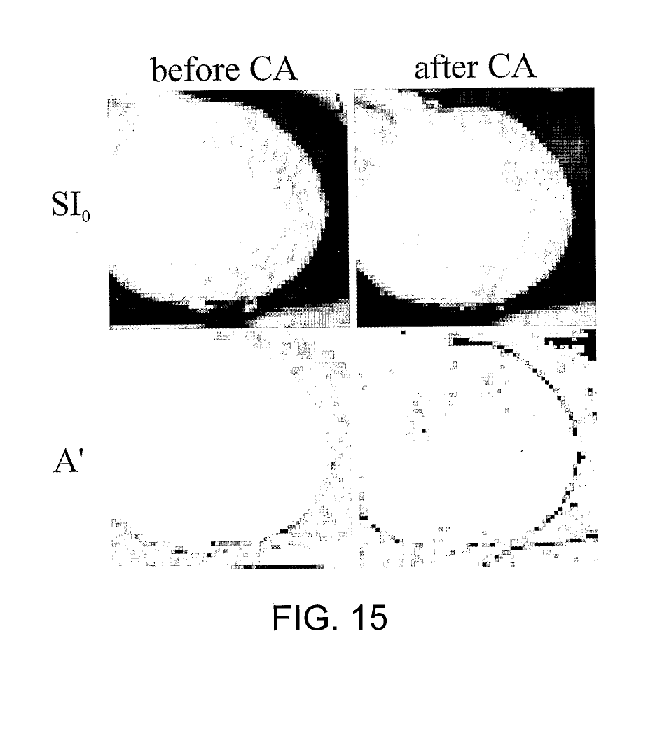

SI₀ and A′ without and with Gd(DTPA): FIG. 15 shows an example of equatorial short-axis SI₀ maps generated in a test dog before and after CA administration using a set of multiple TIs. No significant difference was found between SI₀ values before (141.8±21.8) and 20 minutes after (150.4±22.6) CA administration. This was no surprise, as SI₀ does not depend on the R1 of the tissue voxel, therefore, the administration of a contrast agent indeed should not alter the SI₀ of individual voxels, regardless of the local concentration of contrast agent.

The parameter A′ (the parameter representing the accuracy of the 180° pulse), however, may in principle be influenced by the administration of a contrast agent, because paramagnetic agents alter the magnetic susceptibility of tissue. This may lead to local errors in the accuracy of the 180° pulse, unless the imager readjusts the 180° pulse for each use of the IR sequence. From a large number of experiments, however, experience has shown that A′ varied very little in the myocardium (1.9±0.01), and the administration of the contrast agent did not induce a significant change in A′ (1.9±0.02) (FIG. 15). This is most likely due to the scanner's successful automatic recalibration of the 180° pulse. Thus, even if the susceptibility of the tissue in the imaged region of interest is affected by the presence of a contrast agent, the scanner corrects these slight changes by adjusting the 180° pulse, resulting in practically the same A′ values that were obtained in the absence of the contrast agent. Therefore, it was reasonable to fix the value of A′ to 1.9, and this constant number has been always used in the calculations.

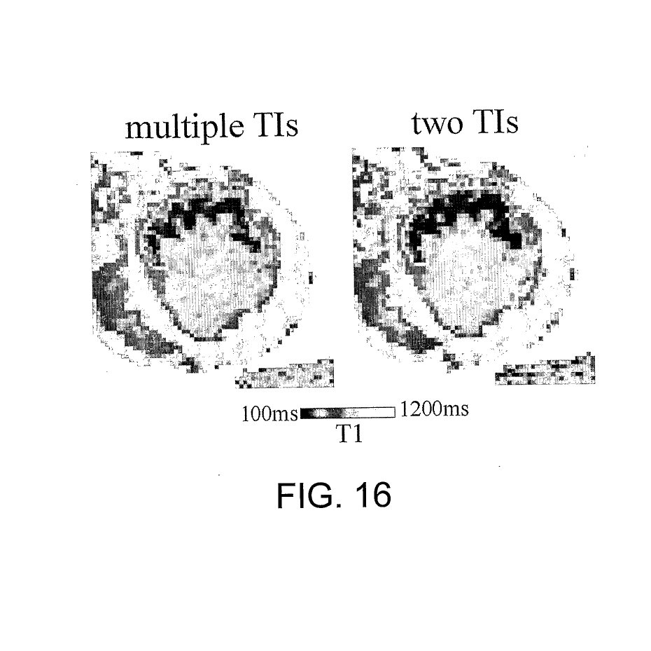

Accuracy of fast R1-mapping using the SI₀ map and only two IR images: To determine the accuracy of T1 (T1≡1/R1) measurement using only two TIs and the baseline SI₀ map, the multiple T1 method was compared to the two-TI method head to head in the same dog. First a full set of IR images were acquired with 6 TIs. T1, SI₀, and A′ were calculated voxel-by-voxel using the automated three-parameter least-squares curve fitting routine. Subsequently, two IR images with two TIs (600 and 800 ms) of the exact same slice position were used to calculate T1, utilizing the SI₀ map (obtained from the multi-TI data), and the above mentioned average A′ value (1.9) as detailed in the previous paragraph. The T1 values from the 6-TI curve fitting were then compared voxel-by-voxel with the corresponding T1 values calculated from using two-TIs and the SI₀ map (FIG. 16). The average underestimation of T1 was 3.4±3.1% (determined from 4602 voxels in total), which is still well within the experimental error of any T1 measurements. Thus the fast method proved to be eminently accurate enough to measure T1 and thus, R1.

### H) Percent Edema Mapping (PEM)

PEM: Percent Edema Map—detection of changes in myocardial water content:

The MRI methods and systems discussed above may be used to map viability without contrast agent or detection of edema due to transient myocardial ischemia. This is useful for screening footprints of ischemic events, myocarditis, infection, inflammation, transplant rejection, and the like. Myocardial edema is also present in a number of other disease states such as after cardiopulmonary bypass, myocardial contusion (due to trauma), hypoproteinemia, and the like.

The MRI methods and systems discussed above may be used to map viability using an R2-enhancing contrast agent (e.g., Dy(DTPA), Dy(ABE-DTTA) or iron oxide-based agents). The amount of agent accumulation could be quantified (e.g., for perfusion of viability studies, and the like). Similarly if iron-oxide labeled stem cells were implanted, for example, the number of cells successfully implanted could be quantified.

Elevation of T2, and therefore the increased SI, may be attributed to increased tissue water content or interstitial edema. This suggests that R2-maps may be used to obtain data and analyze tissues for certain clinical parameters, such as, for example, the presence of edema and to generate maps or other visual representations of the clinical parameter, such as a percent-edema-mapping (PEM). The PEM may be used independently or in combination with other pathology maps. Furthermore, since the changes in R2 are more robust, than R1 changes due to myocardial infarction, the R2 map for the purpose of PEM may be obtained without the need for a contrast agent. To show the feasibility of in vivo high-resolution mapping of myocardial edema, pixel-by-pixel LV R2 maps were generated of five healthy dogs (n=5). T2 maps were generated using a double-IR fast spin echo sequence with eight echo times (TE) in the range of 11.2 ms to 106 ms. R2 was calculated by an automated procedure from the Signal Intensity (SI) vs. TE dependence using a two-parameter curve fitting routine with the following Equation:

SI=SI₀·e^{(-TE·R2)}

where SI₀ is the SI at equilibrium. Variation in SI₀ is mainly controlled by variation in proton density and in magnetic field strengths contributions (both B₀ and B₁). These two factors have great influence on T2-weighted (T2w) signal-intensity, and thus their variations are responsible for the signal inhomogeneities that are inherent to T2w images. SI₀ values within the myocardium ranged from 390 to 1070 (average SI₀ was 695±185), which explained the enormous inhomogeneity of signal even in healthy, intact myocardium. The intrinsic T2 value, however, is insulated from extraneous factors. T2 mapping had also been carried out by other investigators, thus, the results were validated by comparing them to T2 results published in recent literature. The average (n=5) control myocardial and liver T2 values were 53±2.5 ms and 49.6±3.6 ms, respectively. These were in good agreement with other investigators' results from patients at 1.5 T (heart 51.2±4.5 ms and liver 48±9.34 ms).

Next, in vivo Tissue Characterization Maps (TCM) were generated in six infarcted hearts (obtained by a closed-chest 180-min balloon occlusion) in dogs at varying time points following reperfusion. T2 maps, Percent-Edema Maps (PEM) and Delayed Enhancement (DE) images (using GD(DTPA) were generated for the purpose of tissue characterization. Three dogs were sacrificed 4 days after reperfusion to compare in vivo MRI findings in the acute phase of infarction to same-day histology. Three other test dogs were monitored by repeat T2 mapping for 8 weeks to follow the evolution of myocardial edema and the recovery of regional myocardial function.

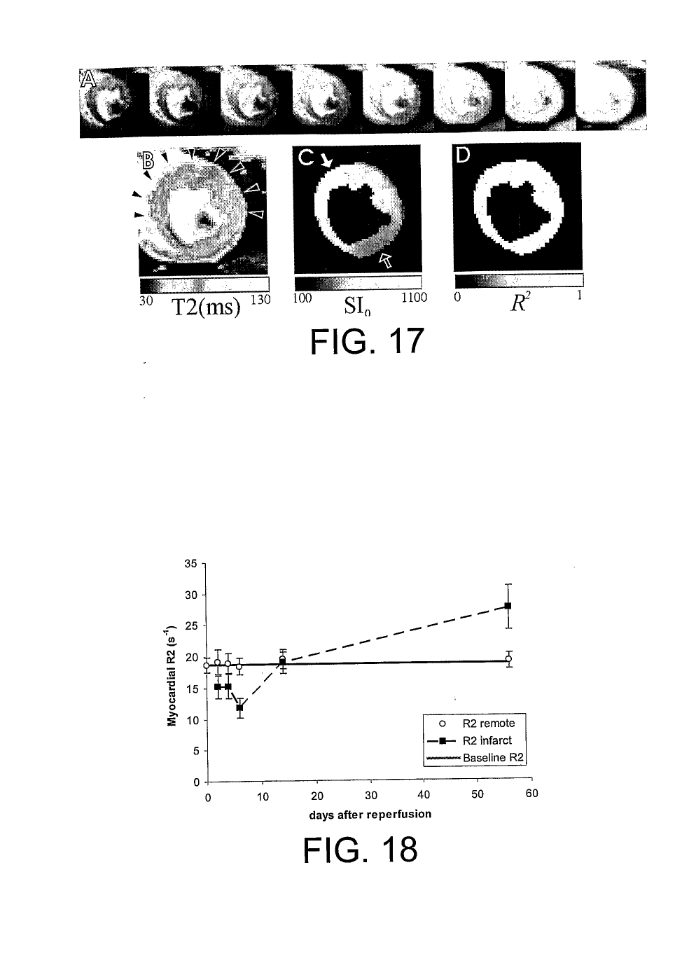

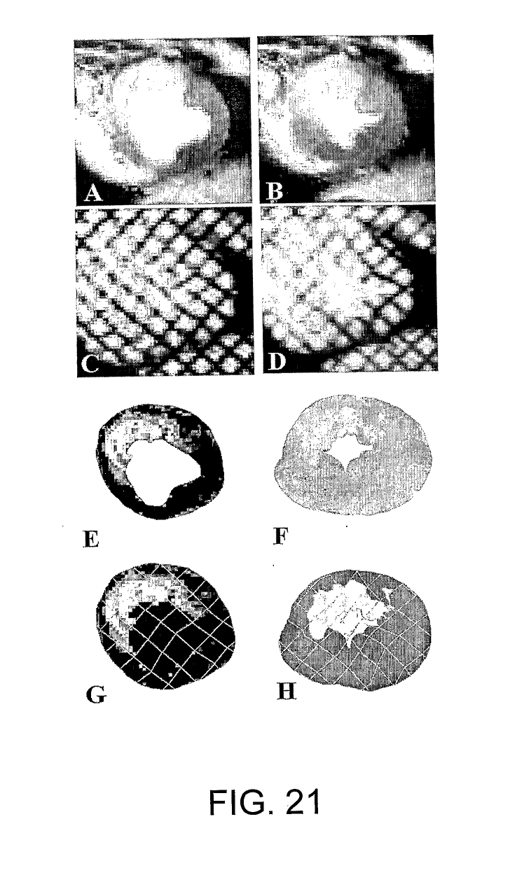

Equatorial short-axis images of a single slice are shown in FIG. 17A with varying TE, obtained for T2-mapping. These images were generated in a dog on day 4 following myocardial infarction. After the imaging session this animal was sacrificed (FIG. 21). Significantly (p<0.01) higher T2 values (FIG. 17B black arrowheads) were found in the infarcted region and in the region neighboring it (67.7±6.6 ms) than in the remote, healthy regions (53±2.5 ms). Large SI₀ variation (FIG. 17C) was observed throughout the myocardium. Although the changes in proton density due to changing water content (inflammation, edema) could yield useful information about the localization and severity of tissue injury, field inhomogeneity also causes large variations in SI₀ among myocardial regions in a manner primarily dependent on their relative position to the receiver coil. Therefore, anterior regions tend to appear brighter (white arrow), while posterior regions tend to appear darker (black arrow). Unfortunately the effects of edema and field inhomogeneity cannot be separated using a T2w image or an SI₀ map.

A voxel-by-voxel T2 map, contrary to a T2-weighted image, shows an intrinsic tissue parameter, and is insulated from many other factors that influence T2w SI (field inhomogeneity, regional variations in proton density, T1 effects, and the like). Myocardial T2 is related to water content, but not in a linear fashion. The transverse relaxation rate R2 (R2=1/T2), however, is linearly related to tissue water content. Thus, R2 is a faithful representation of the magnitude of tissue changes, independent of the pulse sequence or MRI equipment used and is a reliable, reproducible parameter for quantitative calculations.

The accurate coregistration of images obtained with varying TEs was confirmed by the correlation coefficient map (FIG. 17D). This map shows the quality of the non-linear curve fitting (applied to the SI vs. TE dependence) for each individual voxel. The average R² was 0.985±0.01.

From the T2 maps R2 maps (R2=1/T2) were generated and average R2 values were determined in the remote as well as in the infarcted region (outlined by DE). In the long-term dogs, the evolution of R2 values over eight weeks is shown in FIG. 18. A sustained, significant (p<0.01) decrease in R2 values in the infarcted region was observed throughout the first week compared to intact, remote regions (remote R2₀=18.7±1.2 s⁻¹). Lowest infarct R2 was detected on day 6 (11.8±1.6 s⁻¹), which is typically the day of peak edema. Edema retreated by day 14 and R2 returned to baseline. Infarct R2 and remote R2 were not significantly different on that day. Highest R2 was detected in mature scar at 8 weeks (27.5±3.5 s⁻¹). Note that these changes in R2 are more robust than changes in R1 due to myocardial infarction. Therefore, the T2 map for the purpose of PEM can be obtained without the use of a contrast agent. Thus the feasibility of monitoring the tissue evolution of infarcted hearts using in vivo high-resolution myocardial R2 mapping has been shown.

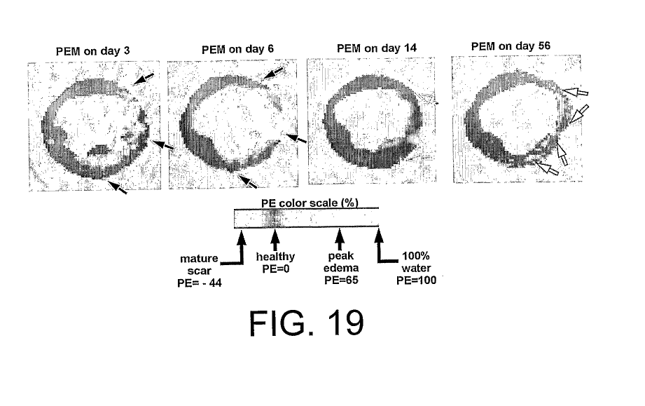

From the R2 maps Percent-Edema-Maps were generated. Representative PEMs generated in another test dog followed for 8-weeks are shown in FIG. 19 at different time points following reperfusion. Edema is clearly apparent in the injured region throughout the first week following reperfusion. Peak edema was detected on day 6 by which time most of the dead myocytes had been cleared away by macrophages (macrophage activity is most intense between days 5 and 7) and granulation tissue was being formed. Note also that the regional wall thickness is greatest at this time point in the affected region. By the end of the second week, the edema almost completely retreated and wall thickness was reduced, and only a residual edema (low percent-edema (PE) values) could be detected in a few voxels within the infarcted region. This was no surprise in light of the fact that no significant difference between infarcted tissue R2 and remote tissue R2 on day 14 (see FIG. 18) was found. Eight weeks following reperfusion, due to the maturation process of scar tissue, PE values in the scar became “negative”, which means that their water content was lower than that of normal myocardium. Regional wall thinning was also observed in these regions due to the shrinkage of the scar tissue. This interesting phenomenon offers yet another use of the PEM method, namely, the detection of mature scar tissue. In patients that suffer reinfarctions, for example, this method would reliably differentiate chronic and acute infarcts, and perhaps even the age of each infarct could be estimated.

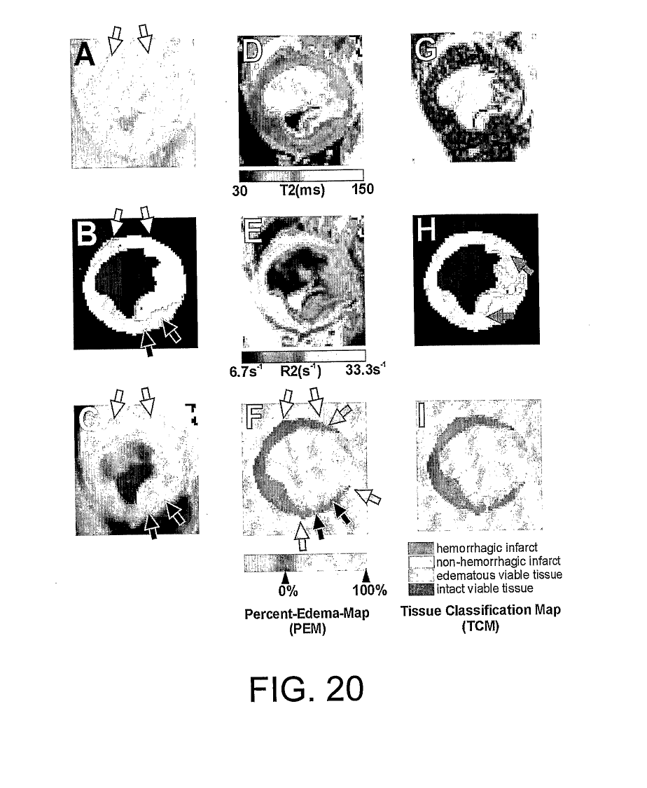

Several important advantages of the PEM method over simple T2-weighted (T2w) imaging exist. FIGS. 20A-F illuminate these advantages. Increased Signal Intensity (ISI) in T2w images has previously been defined as signal intensity (SI) greater than the remote myocardial SI plus 2SD. Thresholding T2w images (FIG. 20B) leads to the overestimation of edematous (enhanced) area in the anterior and septal regions due to the closeness of the coil. In some posterior regions (Black arrows in FIG. 20B), however, edema is not detected with T2w imaging, even though it is present according to the PEM (FIG. 20F). This illustrates the possible underestimation of edema in the posterior regions (far from coil) when using T2w imaging. Since PEM (FIG. 20F) is based purely on an intrinsic tissue parameter, the transverse relaxation rate (R2) (FIG. 20E), it is highly reproducible and standardizable and could be used in large-scale multi center studies. T2w SI, however, is influenced by many extraneous factors and is also highly dependent on the imaging pulse sequence, imaging parameters and the MRI scanner used.

### I) Tissue Characterization Map (TCM)

In the next step, from the PEMs and the corresponding DE images (FIGS. 20G and H), Tissue Characterization Maps (TCM) (FIG. 20I was generated based on the algorithm described herein. The TCM is basically capable of classifying myocardial voxels into the different tissue classes (FIG. 21).

An example for the TCM of a non-hemorrhagic infarct on day 6 following reperfusion (acute phase) is shown in FIG. 20I. It is clearly apparent that the edematous region exceeded substantially the necrotic region highlighted by the contrast agent in the DE image, and that the extent of edema was generally higher (higher PE values) close to the necrotic region than it was farther away, towards the periphery of the Region-At-Risk (RAR). It is worth noting also that the distribution of edema in the TCM (FIG. 20I) is more realistic than the edema detected by the T2w image (FIGS. 20A and 20B). In the TCM, edema completely surrounds the necrotic region, and diminishes gradually with increasing distance from the center of the infarct. In the T2w image, however, it appears that most of the septum is edematous (between white arrows) in spite of the fact that this region is quite far from the actual infarct. On the other hand, in the posterior region, no edema is detected by T2w imaging immediately next to the infarct (black arrows in FIG. 20B), as if there had not been a peri-infarct zone of injured but viable (edematous) tissue surrounding this part of the infarct. These inconsistencies in edema distribution observed with T2w imaging are eliminated by the superior quality of the TCM method.

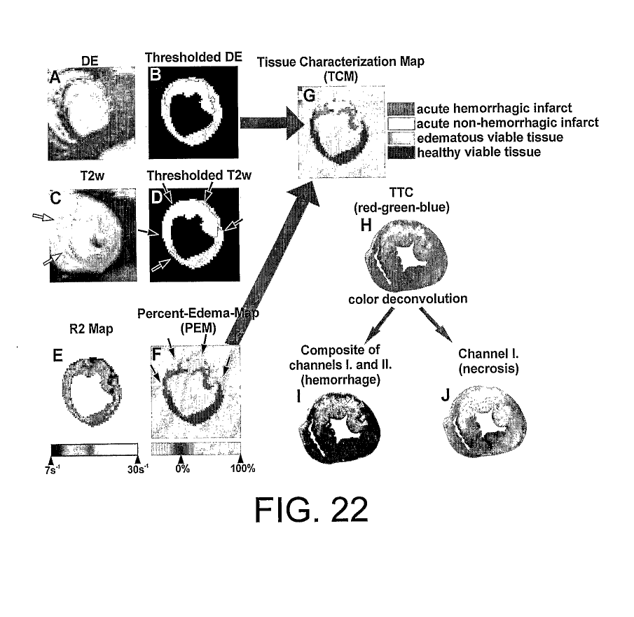

Another example of a TCM, generated in a test dog on day four following a hemorrhagic acute infarct, is shown in FIG. 22G. This figure also shows the processing steps of TCM, as well as the corresponding T2w image for comparison's sake. It is universally accepted that hemorrhage occurs in the center of the infarcted region and is therefore surrounded by non-hemorrhagic but necrotic tissue. Note that the PEM (FIG. 22F) locates the ischemically injured region correctly (the reference being the DE images of FIGS. 22A and 22B). The appearance of the lack of edema in the PEM in the center of the infarct, however, does not represent healthy tissue, but is rather indicative of intramyocardial hemorrhage. The TCM (FIG. 22G), obtained from combining the thresholded DE image with the PEM, is a faithful representation of the distribution of varying tissue types.

When compared to the TTC stained post-processed photo (FIG. 22I), the extent and the localization of infarct and hemorrhage in the TTC photo match those seen in the TCM. Note that the T2w imaging (FIG. 22C) technique is unable to differentiate varying extents of edema, since the gradation in the SI information is lost in the process of thresholding the T2w-image (FIG. 22D). Also, the SI in the anterior region is inherently higher due to the closeness of the coil. Therefore, even hemorrhagic regions display SI values higher than those of the remote myocardium. Thus, T2w imaging is unable to reliably differentiate hemorrhage from edema. It is also clear from FIG. 22F that the T2w method overestimates the area of the edematous region by including the entire septum, in spite of the fact that the intrinsic R2 parameter (FIG. 22E) in most of the septum is not different from the R2 in the remote regions thus indicating absence of edema in the septum.

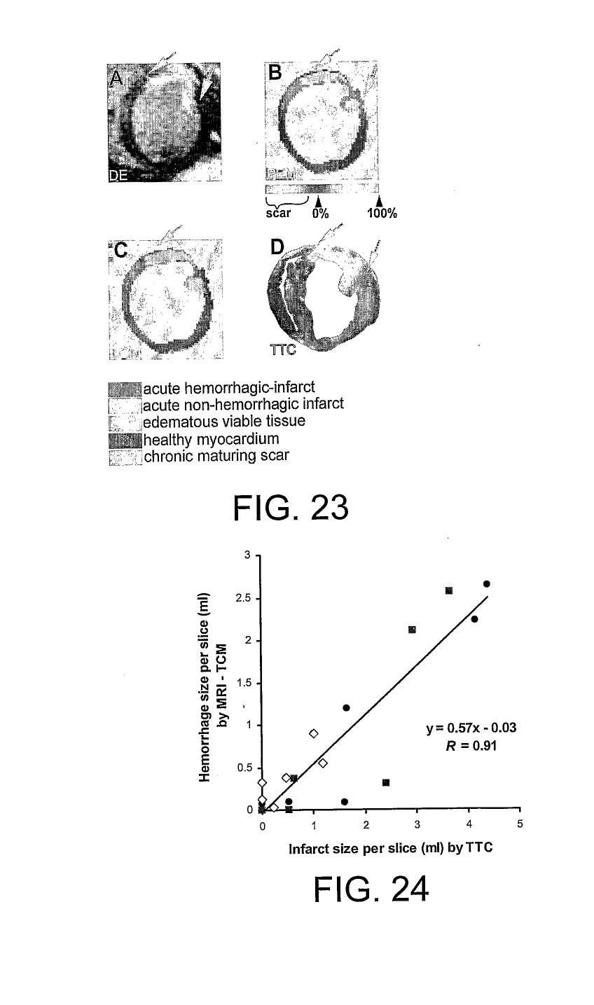

A third example of a TCM generated in a dog 8 weeks following infarct reperfusion is shown in FIG. 24C. The TCM clearly differentiates chronic infarcts from acute ones (compare with FIGS. 22I and 23G). Chronic maturing scar appears as a hyperenhanced region on the DE image (FIG. 23A), and negative edema (water content decreased below normal) on the PEM (FIG. 23B). Depending on the maturity of the scar, water content progressively decreases. Therefore, with the TCM method one could also determine the maturity phase of each individual scar area in the same heart. A second method for confirming that an infarct is exclusively chronic and no recent ischemic injury is present, is checking in the TCM for the presence of edema immediately surrounding the infarct. If the scar tissue is surrounded by edema uniformly then there is a possibility that the infarct has an acute component (reinfarction) (FIG. 22G). If, however, there is no edema surrounding the infarct bed, and the entire necrotic region appears scarred, then recent new ischemic injury can be excluded (FIG. 23C).

Quantification of Hemorrhage: Usually a myocardial hemorrhage occurs following reperfusion and it is known to be a type of reperfusion injury. In the three test dogs that were sacrificed 4 days after reperfusion it was observed that the extent of hemorrhage within the infarct region was linearly related to the size of the infarct. The correlation between the size of the hemorrhage and the size of the infarcted region was examined in all TTC-slices (3.3 mm thick each) of three dogs. A significant (p<0.001) correlation was found (R=0.98) with a regression line of y=0.53x−0.03. The regression line crosses the x-axis at 0.056 ml, a value that can be interpreted as the minimum size of infarct that is coexistent with hemorrhage upon reperfusion. In general, hemorrhage seems to affect about 50% of the infarcted tissue.

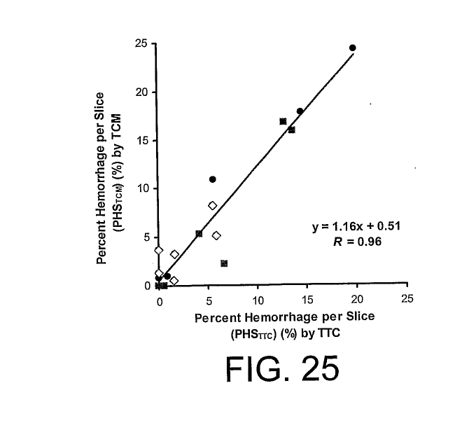

When quantifying the hemorrhage detected in vivo with MRI TCM, the same correspondence was observed between the in vivo hemorrhage size (TCM) and infarct size obtained from the postmortem TTC-photos (note that 3 adjacent 3.3 mm TTC slices were summed to compare with each 10 mm MRI slice). The regression line obtained for the in vivo TCM data (FIG. 24) looked very similar to the one obtained for the histochemistry alone and the correlation was significant (R=0.91, p<0.01). Similarly, a strong correlation (R=0.96, p<0.01)) was obtained for Percent-hemorrhage-per-slice with TCM (PHS_{TCM}) vs. PHS with TTC (PHS_{TTC}) (FIG. 25) with a regression line of y=1.16x+0.51 indicating near-identity of the in vivo and the postmortem PHS results.

These findings strengthen our hypothesis that using the TCM method, the extent of intramyocardial hemorrhage can be quantified accurately. Although no other presently available imaging methods are capable of carrying out such measurements, the TCM method could be easily implemented on any of the presently used clinical MRI scanners.

### J. Edema Score (ES), Hemorrhage Score (HS), Regional Myocardial Function and Remodeling:

Edema and hemorrhage are well known to impede ventricular function. Since the TCM is originally generated with a voxel-by-voxel resolution, any lesser resolution can be derived from it if comparison with results from techniques of poorer intrinsic resolution is required. For example, regional function data are of inherently lower resolution than the voxel-by-voxel resolution of MRI-derived maps. Thus, at times, the latter need to be resolved using some sectoring method that a researcher or clinician deems necessary and appropriate for comparing with similarly sectored regional function data. For the purpose of investigating functional consequences of regional tissue changes, the voxel-by-voxel TCM needs to be divided into sectors with sector borders that are identical to the sector borders used for determining regional function (since, clearly, a function for individual voxels per se is undefinable). For each sector (16 sectors per each short axis slice) an Edema Score (ES) and a Hemorrhage Score (HS) can be derived from the TCMs.

### K. Validation of Our Novel Digital Photo Processing Method for the Evaluation of TTC-Stained Myocardium:

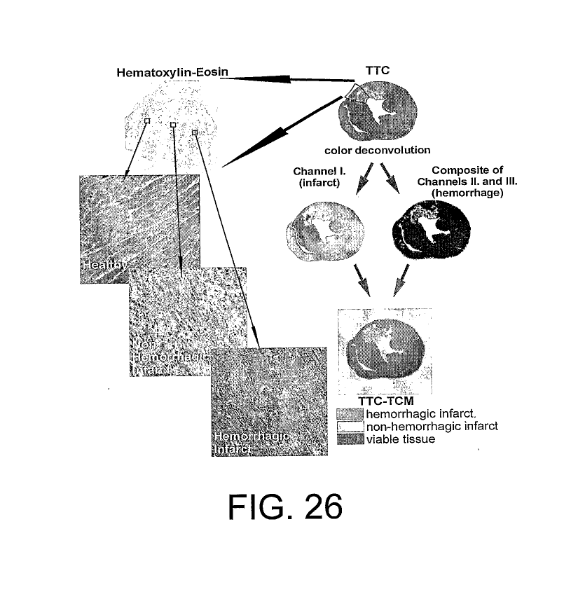

The macroscopic TTC findings were validated with microscopic histology (FIG. 26). Representative samples from the LV wall, where all three compartments (viable, non-hemorrhagic necrosis, hemorrhagic necrosis) were present, were paraffin embedded, sectioned (5 μm), and stained using Hematoxylin-Eosin. The microscopic findings in the three compartments were classical (see caption for FIG. 26), in agreement with the literature. Thus, the TTC-color-deconvolution technique is indeed an accurate macroscopic method for differentiating hemorrhagic, non-hemorrhagic-necrotic and viable myocardium.

### L. Apparent Relaxation Rate-Based Determination of Tissue Clinical Parameters:

In vivo measured (apparent) R1 and R2 values may differ from R1 and R2 values that can be measured ex-vivo. The difference can be attributed to physiological processes. Any change in the intrinsic R1 and R2 parameters, or, the apparent (R1ₐₚₚ and R2ₐₚₚ) parameters, could be used as an indicator of non-physiologic processes or states. In a healthy organ in a certain type of tissue, both the in-vivo and the ex-vivo R1 and R2 are homogeneous (i.e., there is no contrast among tissue segments of the same type). Sometimes, however, physiological processes influence R1 and R2 measurements (e.g., microcirculation, diffusion, and the like). Thus, in a tissue having, or suspected of displaying, different degrees of a clinical parameter, such as acute myocardial ischemia, altered physiological processes (e.g., reduced blood flow in the microcirculation) can change the in-vivo measured R1ₐₚₚ and R2ₐₚₚ values locally, even if on ex-vivo imaging the R1 and R2 values would be homogeneous in all areas. This, in turn, allows the detection of even such subtle changes.

An example for the use of this technique would be the generation of serial MRI images of the heart following a selective 180° pulse (applied to the imaging plane) using variable inversion times (TI) and timing the acquisition of data to the end-diastolic phase (to eliminate motion artifacts and to improve spatial co-registration of tissue regions). After applying non-linear curve fitting to the SI vs. TI dependence, the measured R1ₐₚₚ would be the sum of the intrinsic (ex-vivo) R1 plus the R1_{microcirc} (R1ₐₚₚ=R1+R1_{microcirc}) attributed to non-inverted spins (SI=SI₀) entering the inverted region of interest due to microcirculation and diffusion of water molecules. Such information could be used to determine with great accuracy a blood flow map of the microcirculation of a tissue of interest, which is not currently done.

Localized 180° pulses within the imaged area could also be used for the same purpose, similar to the tagging cine method used for assessing regional function. Images could be collected serially while the heart is moving (similarly to the known Look-Locker method). With the spatial guidance provided by the tagging grid, the movement of tissue regions of interest could be traced. In this method, however, not the movement of the tissue, but the SI change with varying inversion times would be studied. The same non-linear curve fitting method would yield the R1ₐₚₚ values of these regions of interest.

Note that this method may be applied with or without a paramagnetic contrast agent or other means of contrast enhancement (e.g., a cold isotonic saline bolus would cause a change in detected R1 due to the relationship between temperature and R1).

### M. Increasing Spatial Resolution by Frame-Shifted R1 or R2 Maps:

The trade-off of decreasing slice-thickness (and consequent increasing of spatial resolution and reduction of partial volume effects) in MRI imaging is the loss of signal-to-noise-ratio (SNR). Utilizing the averagable property of relaxation rates (such as R1 and R2) the effective spatial resolution of MRI could further be increased without reducing SNR.

For example, a slice with thickness S=2 s is used in this illustrative example. After acquiring R1 (or R2) data on a first slice with a thickness (S) that allows a good SNR, the spatial position of the tomographic slice is shifted by a distance “s” along a line perpendicular to the imaging plane to acquire R1 (or R2) data on a second slice (having a thickness of S) such that this newly prescribed slice partly overlaps the previous slice. The same R1 (or R2) data collection can be carried out in this shifted position. Finally in the original position, data is obtained from a slice with a thickness of S+s to determine a starting point for the calculation (this slice essentially includes the volume of the first and second slices).

The difference between the R1 (or R2) values of this last slice and the second, (shifted) slice would yield the ΔR1 (or ΔR2) values of the non-overlapping part of the first slice (i.e., the part of the first prescribed slice that is not overlapped by the second slice) relative to the second slice. Note that the R1 contribution values of subvoxels with the shifting method should be calculated with weighting for proton density (or if proton density is assumed constant then volume can be used for weighting instead). An alternative weighting factor can be the above mentioned SI₀ as well.

Thus, effective tissue information from a thin slice, with a desired thickness equal to the shift applied (s), could be obtained with a SNR that is equal to that obtainable with an optimal slice thickness (ST). Appropriate visual representations or maps could be generated using these values as described herein. The same principle may be applied to SI values as well.

### N) PPeM: Percent Perfusion Map:

Pixel-by-pixel hi-res quantitative perfusion mapping can be carried out by measuring the R1 value in a given voxel after the administration of a contrast agent that distributes to the tissue in proportion with regional blood flow (RBF). By knowing the maximal R1 enhancement in a well perfused region (percent perfusion (PPe)=100%) and knowing the baseline R1 value of the tissue in question (PPe=0%) a quantitative scale can be determined that expresses RBF in terms of the measured R1 values.

### O) Other Combinations of the Parametric Maps:

The tissue clinical parameter maps disclosed herein may be combined as desired to derive new clinical parameters. For example the PIM may be combined with the PUM to generate a map that shows infarct regions that are underperfused.

Another example is the PIM, which is calculated from the increase in R1 values due to contrast agent (CA) accumulation in infarcted tissue. Regional edema, however, has an effect that is opposite to that of the CA (i.e., edema decreases R1). If, on the other hand, the regional water content is mapped using PEM, the baseline R1 values may be corrected for edema, leading to more accurate measurements

### P) Improved Method of MRA:

If ΔR1 is known for the myocardium (in a region where there are only capillaries and no macroscopic vessels) then the additional ΔR1 detected can only be attributed to the presence of vessels, which may even be smaller in diameter than the voxel. Thus, based on ΔR1, the vessel volume in each voxel could be quantified, from which the diameter of the vessel could be calculated and eventually a high-spatial-resolution MRA could be reconstructed with the help of the “gross” MRA, and vessel diameter could be assessed more accurately and even slight stenoses may be revealed and even small branches may be detected.

### Q) Total Vascularization:

Blood volume could be quantified based on the actual R1 value as long as a contrast agent that is 100% intravascular is used and the baseline R1 is known of a given tissue without blood in it. Thus, all the R1-enhancement following CA administration would be due to the enhancing effect of the CA. Although the intrinsically lower R1 of the blood decreases the overall effect of the CA, it does so in proportion with the blood volume. Thus, this confounder factors out.

### R) Total Organ R1 Measurements:

Using NMR Spectroscopy of a large selected volume and calculating global infarction fraction from the global ΔR1 values could be possible. Similarly, prostate global neoplasm fraction could be determined. Another example is whole-brain measurement of R1 or R2 could detect brain tumors, various kinds of diseases (e.g., PQM of Huntington's disease; Q=glutamine) or could be used to monitor treatment (e.g., tumor size) or disease progression (e.g., multiple sclerosis).

A further use of this technique would be to noninvasively quantify various in vivo blood parameters (e.g., noninvasive Hematocrit and Hemoglobin content based on R1 difference of plasma vs. fully Hb-loaded RBC)

### S) Persistent Hyperenhancement Using the Standard Gd(DTPA) Dose but Administered in Continuous Infusion:

A small bolus of Gd(DTPA) (or other contrast agents with fast-kinetics), followed by continuous supplementation, may enable us to carry out R1 mapping and to generate PIM. Slow, continuous administration of GD(DTPA) allows the accumulation of GdDTPA in the infarct and a prolonged R1 contrast, and create steady state kinetics, where R1 mapping can be carried out without a significant change in R1, as the wash out is compensated for by the slow administration of the agent.

### T) Virtual Biopsy Maps in the Prostate:

Based on the fact that the PCA is distributed in the prostate containing prostate intraepithel neoplasia in inverse proportion to the cancerous tissue concentration in the TRAMP mouse model a virtual biopsy map can be generated from the R1 map. The R1 value of a given prostate volume element is governed by the fraction of neoplastic tissue in that volume element. Therefore, an R1 map made following the administration of PCA (or any contrast agent showing similar tissue characteristics) similarly to the method of Percent Infarct Mapping, can differentiate a combination of normal prostate tissue and low grade mouse prostatic intraepithelial neoplasia (mPIN) from a combination of tissues with high grade mPIN and well differentiated adenocarcinoma (WD) within the murine prostate lobes. Therefore, our method yields an in-vivo-obtained virtual biopsy map (VBM). In this manner the 3D information inherent in MRI images can be used to quantify the extent of neoplasm.

### Calculations, Procedures, and Methods

Note that the methods described here are for the purpose of giving examples and not for the purpose of limiting the methodologies of the present disclosure. In fact these methods may be applied to many other methods (even non-MRI) that acquire raw imaging data.

Canine Model of Reperfused Myocardial Infarction:

In the canine model used in the present disclosure, the myocardial infarct was generated by prolonged proximal occlusion of the Left Anterior Descending coronary artery, using an intracoronary balloon catheter. Animals were anesthetized and mechanically ventilated during MRI sessions (using isoflurane).

MRI

A 1.5 Tesla GE Signa Horizon “Cardiac CV” instrument was used for cardiac MRI. Images were generated in standard consecutive short-axis planes (6 slices covering the entire LV) and one additional three-chamber long-axis view to assess the apex (left ventricular outflow tract view).

R1 Mapping:

A 180°-prepared, segmented, fast gradient-echo sequence, was used to determine R1 values of the myocardium using the inversion recovery method. Short axis and long axis images were generated using the following parameters: FOV 300 mm, image matrix 256×256, slice thickness 10 mm, read out flip angle 25°, echo time (TE) 3.32 ms, repetition time (TRp) 7.18 ms, and recycle time (TR) 1200-2000 ms (three R-R intervals, depending on heart rate, but constant throughout an entire given R1 mapping procedure). Inversion times (TI) in the range of 200-1200 ms were used. In theory, 6 different TIs should be sufficient to generate a R1 map. In this disclosure, however, 8-10 TIs were used to ensure accuracy of R1 determination. The R1 values were calculated from the SI vs. TI dependence, applying a three-parameter least-squares curve fitting routine, using the following formula:

SI=SI₀(1−A·e^{-T1·R1}+e^{-TR·R1}) [1]

where SI is the signal intensity observed at a given inversion time (TI) and SI₀ is the signal intensity at equilibrium and A is a parameter (A≦2) dependent on the accuracy of the 180° inversion pulse. Magnitude signal polarity was assigned as described by Nekolla et al. (Nekolla S, Gneiting T, Syha J, Deichmann R, Haase A. T1 maps by K-space reduced snapshot-FLASH MRI. J Comput Assist Tomogr. 1992; 16:327-32.

Since all MRI parameters below reflect the entire volume element in the slice, associated with a given pixel, from this point on the term voxel, rather than pixel, will be used.). The relaxation rate R1 (R1≡1/T1) of each voxel was composed of R1₀, the myocardial R1 observed in the absence of both infarct and PCA, and ΔR1, the contribution of the PCA present in the voxel:

R1=R1₀+ΔR1 [2]

Using Eq. 2, the R1 map was transformed into a ΔR1 map. The average ΔR1ᵣ value of areas remote from the infarct zone (about 600 voxels), was considered representative of 0% infarction.

ΔR1_{c}=R1_{c}−R1ᵣ [3]

The infarct-based ΔR1 of each voxel, ΔR1ᵥ was obtained as follows.

ΔR1ᵥ=R1ᵥ−R1ᵣ [4]

As a threshold, ΔR1ᵥ of all remote voxels where R1ᵥ<R1ᵣ+2SD, was set to zero. After localizing the infarcted area, the center of the infarct core (5-10 voxels), and which displayed the largest ΔR1, was identified and its ΔR1ᵥ was chosen as representative of 100% infarction (ΔR1_{c}).

ΔR1_{c}=R1_{c}−R1ᵣ [5]

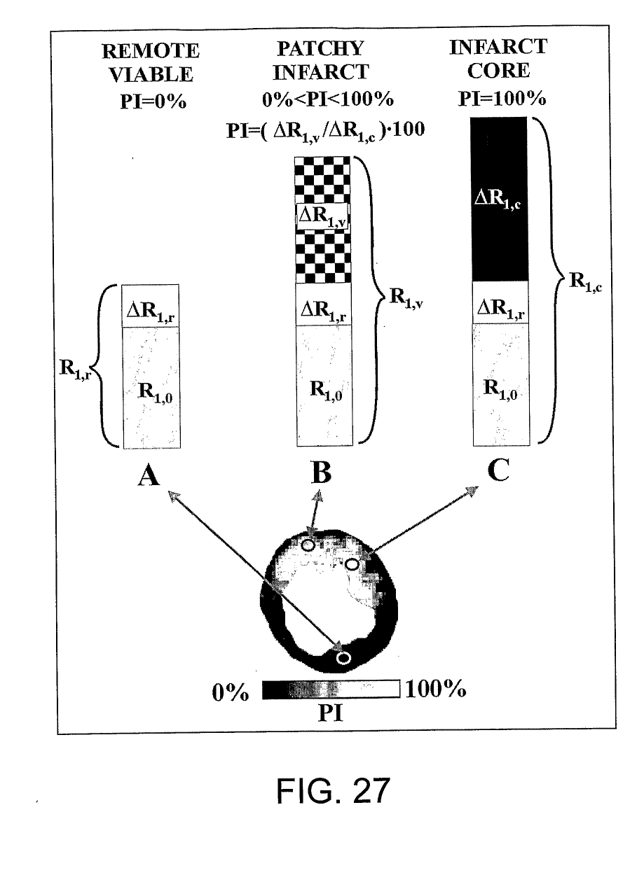

Solid infarcts in these regions were later confirmed postmortem by examining the corresponding regions on both sides of each of the three TTC-stained 3.3 mm slices. In all cases these regions were found void of any viable tissue. Subsequently, a percent-infarct (PI) scale between 0% and 100% infarct was generated in terms of ΔR1ᵥ values. PI values were then calculated from ΔR1ᵥ values (FIG. 27):

PIᵥ=(ΔR1ᵥ/ΔR1_{c})·100 [6]

where PIᵥ was the PI of a given voxel.

Substituting ΔR1ᵥ from Eq. 4. and ΔR1, from Eq. 5., Eq. 6. became:

PIᵥ=(R1ᵥ−R1ᵣ)/(R1_{c}−R1ᵣ)·100 [7]

Thus, the R1₀ value was not needed for the calculation of percent infarct values. The PI value for any voxel could be calculated from the actual R1ᵥ using the actual post-contrast R1ᵣ of remote, viable areas and the R1_{c} measured in the infarct core.

Color coding and the 3D reconstruction of the PIM were carried out using ImageJ (Wayne Rasband, NIH). Thus, no sharp infarct contours were forced. PI-per-slice (PIS_{PIM}) was determined by averaging all PIᵥ values in a given LV slice.

Accuracy of PIM Calculation:

The practical sensitivity of the PIM method can be calculated as follows. ΔR1_{c} is the relaxation rate enhancement attributable to 100% infarction. Thus, if 1% of myocytes in a voxel were infarcted, the R1-enhancement obtained relative to healthy areas would equal 0.0121 s⁻¹ and 0.0127 s⁻¹ at 24 h and 48 h, respectively. The average SD of remote R1ᵣ within individual experiments was 0.06 s⁻¹. Since a 2SD cutoff was used in our calculations to define PI=0, infarcted tissue may have remained undetected only in voxels where PI≦0% (2·0.06/0.0121) and PI≦9% (2·0.06/0.0127), at 24 h and 48 h after PCA, respectively. In other words, only infarctions that affected less than 10% of cells in a voxel could have remained undetected with PIM. Thus, the threshold of infarct detection with PIM was 1.37 mm³ (10% of voxel size).

Slice Percent Infarct (SPI):

Once the PIM was obtained, in each slice endo and epicardial contours were traced and PIᵥ values were summed in all myocardial voxels.

SPI_{PIM}=Σ(PIᵥ·Vᵥ)/Σ(·Vᵥ)·100 [8] (summation over all myocardial voxels v in the slice)

where SPI_{PIM} is the percentage of tissue infarcted in any given slice, and Vᵥ is the volume of a voxel.

Global LV Infarction Fraction (IF):

A global LV infarction fraction (IF) was also calculated:

IF_{PIM}=100·Σ(PIᵥ·Vᵥ)/Σ(·Vᵥ) [9] (summation over all myocardial voxels v in the heart)

where IF_{PIM} is the infarction fraction of the LV determined from PIM

R2-Mapping:

R2 values were determined by double-IR prepared fast spin echo images acquired with varying Echo Times (TE) in the range of 10-100 ms. The following parameters were used: FOV 300 mm, image matrix 256×160, slice thickness 10 mm, flip angle 90°, Echo Train Length=24, and recycle time (TR) 1000-1400 ms (depending on heart rate, as the number of R-R intervals used was 2, but constant throughout an entire T2 mapping procedure). A non-linear least squares curve fitting routine was applied to the SI vs. TE dependence to calculate R2.

To calculate R2 (R2≡1/T2) the following formula was used:

SI=SI₀·e^{-TE·R2} [10]

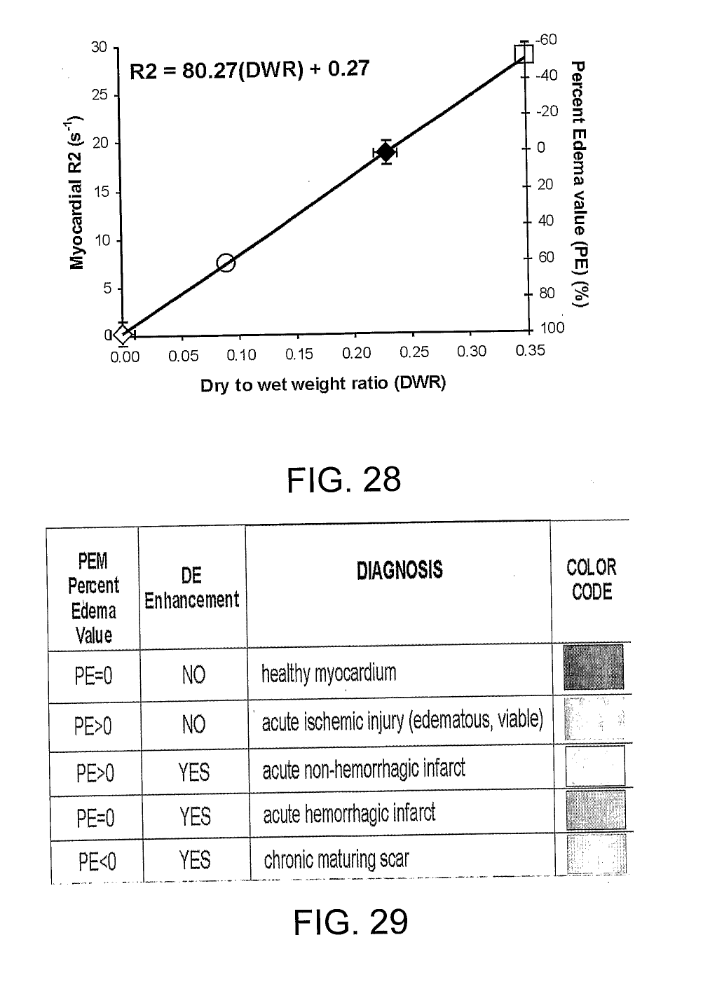

### ΔR2 and percent-edema (PE) were calculated as shown in FIG. 28.

Generating Baseline SI₀ and R1₀ Maps:

Non-contrast-enhanced R1₀, and SI₀ values will be calculated, from the SI vs. TI dependence, applying a non-linear, three-parameter least-squares curve fitting routine, using Equation [1] exactly as above for contrast-enhanced R1 maps.

The value of SI₀ is governed to a large extent by proton density in the given voxel, magnetic field inhomogeneity, and other confounders influencing the magnetic field. SI₀, practically changes from voxel-to-voxel throughout the imaged area and is a significant component of the detected SI value but independent of the imaging parameters (TI, TR) used and independent of the intrinsic R1 parameter. SI₀ is also a reflection of the experimental conditions at the time of imaging, including effects of field inhomogeneity, the physical properties of the MRI equipment, coil position, patient position at the time of imaging, and the imaging sequence and parameters used.

Post-Contrast R1 Mapping Using SI₀ and T1-Weighted Images:

Since SI₀ does not depend on the R1 of the tissue voxel, and is also independent of other imaging parameters used (TR, TI or the accuracy of the preparation pulse), the administration of a contrast agent does not alter the SI₀ of individual voxels, regardless of the local concentration of the contrast agent.

The parameter A′, however, may be influenced by the administration of a contrast agent, because paramagnetic agents may alter the susceptibility of tissue. Theoretically, this may lead to local errors in the accuracy of the 180° pulse. From a large number of experiments, however, our experience had been that A′ had very little variability in the myocardium (1.9±0.01), and the administration of the contrast agent did not induce a significant change in A′ (1.9±0.02). This is due to the fact that the MRI scanner, before the acquisition of each IR image set, automatically recalibrates the 180° pulse. Thus, even if the susceptibility of the tissue in the imaged region of interest is changed by the presence of a contrast agent, the scanner corrects these slight changes by adjusting the 180° pulse, resulting in practically the same A′ values that were obtained in the absence of the contrast agent. Therefore we fixed the value of A′ to 1.9, and this constant number was used in our further calculations.

Knowing SI₀ (determined individually for each voxel in the baseline), A′ (equals 1.9, as determined empirically from a large number of experiments for a given scanner), and TR (calculated from the heart rate at the time of imaging and the number of R-R intervals used as recycle time), allows the determination of a family of relaxation curves that model in a given voxel the SI vs. TI dependence for all possible R1 values. What remains is to sample from this family of curves the one that yields the actual R1 for that particular voxel.

Following the administration of the CA, an IR image is acquired with a single TI (or a few TIs). The SI value(s) obtained for a given voxel with the selected TI(s) allows the sampling of the virtual pool of relaxation curves for this voxel (determined by SI₀, A′, TR and R1) and the selecting of the curve that best agrees with the SI value(s) measured at the given TI(s). The R1 that fits this curve yields the actual R1 value.

The accuracy of this method was tested as follows. First a set of IR images were acquired with 10 TIs. R1, SI₀, and A′ were calculated voxel-by-voxel using a three-parameter least-squares curve fitting routine. Then, IR images obtained with two TIs (600 and 800 ms) in the exact same position were used to calculate R1, utilizing the SI₀ map, and the above mentioned average A′ value (1.9), as detailed above. The R1 values from the 10-TI curve fitting were then compared voxel-by-voxel with the corresponding R1 values calculated from using two-TIs and the SI₀ map. The average underestimation of R1 was 3.4±3.1% (determined from 4602 voxels), which is still well within the general experimental error of R1 measurements by any technique. Thus the method proved to be accurate enough to measure R1.

Percent-Edema-Map (PEM) calculations: The fast spin echo image sets will be processed, and R2 (R2≡1/T2) will be calculated voxel-by voxel, from the TE dependence of the SI, by means of a two-parameter, least-squares curve-fitting routine, using the following formula.

SI=SI₀·exp^{(-TE·R2)} [11]

where SI₀ is the signal at the theoretical TE=0 time point and it also represents the maximum SI.

The change in the transverse relaxation rate (ΔR2) induced by tissue changes, and subsequently percent-edema (PE) values (see below), will be calculated from the R2 results as follows.

The direct linear relationship between dry-to-wet weight ratio (DWR) and myocardial R2 following myocardial infarction has been shown. Based on this linear relationship, the changes in R2 (ΔR2) can be interpreted as changes in water content.

FIG. 28 shows this relationship between R2 and DWR at 1.5 T. Since an increase in tissue water content leads to a decrease in tissue R2, we defined ΔR2 in such a way that we obtain a positive value when water content increases.

ΔR2=R2₀−R2 [12]

where R2₀ is the R2 measured in healthy myocardium (black diamond), and R2 is the actually measured R2 in any given (unaffected or affected) myocardial region. Thus, in unaffected myocardium ΔR2=0 (PE=0), in edematous regions ΔR2>0 (positive PE values), and in regions, where the water content is decreased (mature scar) ΔR2<0 (negative PE values).

To determine a universal percent-edema (PE (%)) scale in terms of ΔR2 values, the value of PE=0 was assigned arbitrarily to the healthy, edema-free myocardium, where ΔR2=0 by definition (black diamond). R2 maps were generated in five healthy control dogs and obtained an average R2₀ value of 18.7±1.2 s⁻¹. The corresponding DWR in such normal myocardium was 0.23±0.01.

PE=100 was assigned to the ΔR2 of pure water, which is the theoretical maximum (though practically unattainable) tissue water content and thus it corresponds to a DWR of zero by definition (open diamond). The R2 of pure water is R2_{H2O}=0.27 s⁻¹. ΔR2 of pure water can be calculated as follows:

ΔR2_{H2O}=R2₀−R2_{H2O}=18.7−0.27 s⁻¹=18.43 s⁻¹ [13]

This value is the change in R2 when proceeding from healthy myocardium (DWR=0.23) to pure water (DWR=0). This ΔR2_{H2O} corresponds to the entire theoretical range of change in water content covering the corresponding range of PE from PE=0 to PE=100. Thus this value will later be used to convert any observed R2 to the corresponding PE value of an individual voxel (see below).

Thus, a regression line is determined by these two well-defined PE points (0 and 100) for our experimental model, which can be used to determine water content (in other words, the PE value) in vivo, based on measured R2 values.

To demonstrate the dynamic range of possible observed R2 following a myocardial infarction, two extreme points on this regression line are shown, namely, the R2 of peak myocardial edema (gray circle, lowest detected myocardial R2=7.4 s⁻¹) and the R2 of mature scar (gray square, highest detected myocardial R2=28.6 s⁻¹), which correspond to PE values of 61, and −54, respectively (FIG. 28). Note that due to the decreased water content (increased DWR) of mature scar tissue, R2 values are greater than those in healthy myocardium. Equation [3], therefore, yields negative ΔR2 values for voxels that are scarred, thus, on the PEM, these regions can be identified as regions with “negative” (relative) edema (PE<0). Note that this simply means that, relative to healthy myocardium, these tissue voxels have a reduced water content.

Thus, to calculate the PE values for all myocardial voxels, any given ΔR2ᵥ will be calculated for each voxel v by subtracting the observed R2 value of that voxel (R2ᵥ) from the R2₀ value, measured in healthy regions, remote from the region of the ischemic insult:

ΔR2ᵥ=R2₀−R2ᵥ [14]

In this manner, the R2 map will be transformed into a ΔR2 map. To set a threshold, we set to zero the ΔR2ᵥ of all voxels where (R2₀+2SD)<R2ᵥ<(R2₀−2SD).

Subsequently, a PEᵥ value will be calculated for the given voxel v on the basis of the ΔR2ᵥ determined for that voxel, using the following equation:

PEᵥ=(ΔR2ᵥ/ΔR2_{H2O})·100 [15]

The collection of the individual PE values from all voxels constitute the PEM. Thus, a 60×60 matrix of PEM will be obtained for each slice. Color coding and a 3D reconstruction of the parametric images will also be carried out, using Image J.

Definition of myocardial sectors and the Edema Score (ES): Since myocardial function cannot be quantified with a per-voxel resolution, it is necessary to divide the myocardium into sectors to assess regional function. For the purpose of studying the effect of varying degrees of edema on regional myocardial function, the voxel-by-voxel PEM needs to be resolved using the same sectoring method that is used to obtain regional function data. Since the PEM is originally generated with a voxel-by-voxel resolution, any lesser resolution can be derived from it if comparison with results from techniques of poorer intrinsic resolution is required.

Endo-, and epicardial contours will be traced manually on the parametric R2 maps. The short axis slices will be divided into 16 circumferential sectors in each slice, starting at the posterior interventricular groove and proceeding towards the septum. In the long axis LVOT image, the apical part of the left ventricle not covered by the short-axis slices will be delineated and will serve as the apical sector. Contours will then be transferred to the PEMs. Note also that the same sectoring will be carried out for TCM (see below) as well as for cine MRI images, all to achieve accurate coregistration among the different imaging methods.

In each sector, the PE values of all voxels will be averaged to obtain the severity of ischemic injury per sector. This will be called the Edema Score (ES).

ES_{PEM}=(ΣPEᵥ)/n_{sector} [16]

where n_{sector} is the number of voxels in the given sector.

Tissue Characterization Maps (TCM):

Since both the PEMs and the DE images will be acquired with the same angulations and slices and in the same end-diastolic phase of the cardiac cycle, they will be virtually superimposable. An algorithm to generate elaborate Tissue Characterization Maps with voxel-by-voxel resolution has been produced. The algorithm (FIG. 29) combines the information from the PEM and the thresholded DE image to generate a composite, color-coded image that displays tissue characteristics (FIGS. 21-23). Tissue characterization will be based on the presence or absence of edema, or the presence of “negative edema” (reduced water content) while at the same time taking into account whether or not the voxel is enhanced in the DE image.

Applying this algorithm, a computer routine will generate TCMs based on the voxel-by-voxel PEMs and DE images, by assigning specific values to each of the tissue classes. Color coding will be carried out in ImageJ.

Two examples of TCMs are shown, in FIG. 22 for an acute, hemorrhagic infarct, and in FIG. 23 for an old scarred infarct. Note that the TCM clearly differentiates hemorrhage from non-hemorrhagic necrosis, and acute infarct from chronic infarct. When looking at an in vivo acquired TCM, therefore, the in vivo “histologic” diagnosis can be made at a glimpse, whether the infarct is acute or chronic, hemorrhagic or non-hemorrhagic.

It is important to note that while in healthy myocardium “PE=0” means the absence of edema in the tissue, in hemorrhagic infarction edema is present but is not seen by the PEM due to the masking effect of the susceptibility-based change in R2, induced by met-hemoglobin. This latter R2 change is opposite in direction to the change induced by the increased water content in the edema. This may lead to mutual cancellation.

Quantification of Region at Risk and Hemorrhage Using the TCM:

The Region-At-Risk (RAR_{TCM}) will be measured in each TCM by counting all voxels that are not healthy myocardium, and expressing the voxel count as a percentage of the total voxel count in that slice. This will be compared to the postmortem measurement of RAR_{μφ} using fluorescent microspheres (see below). In the acute phase of the infarction (first week), hemorrhagic infarct areas will be quantified per slice and per sector. A Percent-Hemorrhage-per-Slice (PHS_{TCM}) value will be determined and compared to the hemorrhage seen on postmortem TTC-stained slices (PHS_{TTC}, see below).

TCMs will also be sectored using the same method that was described above for PEM. A Hemorrhage Score (HS) will be determined for each myocardial sector (count of hemorrhagic voxels per sector expressed as a percentage of all voxels in that sector). Regional function, as well as long term recovery of function will be examined separately in these sectors to elucidate the effect of hemorrhage on the recovery outcome.

TTC Staining Method:

TTC staining has been used as a post mortem gold standard to quantify myocardial infarction. It was used to validate the infarct size and location observed and quantified in the MRI images. Immersing the slice in the TTC solution following the freezing of the heart causes distortion. Thus the comparison between the PIMs, calculated from the end-diastolic MRI images, and the photographs of the corresponding TTC-stained physical slices, was problematic. To improve the correspondence the following changes have been made in the procedure.

Instead of immersing the frozen heart slices in the TTC solution, the staining was performed in-vivo, prior to the arrest of the heart. Following the last MRI session, the experimental animals, still anesthetized, were given TTC-containing saline. In some cases the administered solution caused left ventricular fibrillation. Therefore, after the last MRI session left thoracotomy was carried out on the dog, still anesthetized, to expose the heart.

A solution of 12.5 mL/kg of 2% TTC saline was then administered intravenously. To achieve sufficient staining of the living myocardial tissue, this solution had to remain in the circulation at least for 20 minutes. The animal was then euthanized with a high dose of Pentobarbital followed by 100 mL of 2 M KCl solution. In cases where left ventricular fibrillation occurred during this 20 minute period, the circulation was maintained by direct manual massage of the heart.

The heart was then excised and frozen by immersing it in −80 C.° ethanol. Once frozen, the hearts were sliced transversally (3 mm slices). Both sides of each TTC slice were photographed. The volume of the infarcted tissue in each TTC slice and the total LV volume of that slice were determined using Image J, and talking the applied slice thickness into consideration. Hemorrhagic areas appeared dark brown in these images, but they were always located in the center of the infarcted area, and thus could easily be identified. These areas were also included in the measurement of infarcted area. SPI_{TTC}, IF_{TTC}, IVS_{TTC} and IVH_{TTC} were calculated for comparison with MRI data by summing infarcted tissue volumes and myocardial volumes in three TTC slices that correspond to the MRI image, and carrying out the calculations detailed above.

TTC staining is also capable of differentiating three tissue types in the myocardium. These three are viable tissue (stains brick-red), non-hemorrhagic necrosis (appears pale) and hemorrhagic necrosis (appears purple-brownish in the center of the infarct). The digital camera records three channels (RED-GREEN-BLUE) from which a composite color image is reconstructed that looks similar to what we see with our naked eye. Hence only channels I, II, and III are referred to here. Each of these channels record specific wavelength ranges of the spectrum, originating from various tissue types.

Channel I best represents tissue viability, and is useful in delineating infarct borders. Channels II and III contain specific visual information about the intramyocardial hemorrhage. Splitting the three channels of the original photo, therefore, allows to selectively highlight and quantify certain tissue characteristics. For this purpose we developed a TTC-photograph-processing method (FIG. 27) which is similar to the color deconvolution technique described elsewhere and used for evaluation of histochemically stained specimens.

After splitting the three channels, we display channel I, as a grayscale image, where viable is shown as dark grey, and irreversibly injured, necrotic regions are shown as bright (hemorrhage appears as dark grey in the center of the pale region). Infarct borders are then traced on these images using Image J, including both the hemorrhagic and non-hemorrhagic regions. The volume of the infarcted tissue in each TTC slice and the total LV myocardial volume of that slice will be determined by measuring the areas, and taking the applied slice thickness into the calculation.

To highlight hemorrhage selectively, channels II and III will be merged separately. This results in a post-processed composite image, where we show hemorrhagic regions as light brown regions within the greenish-yellow non-hemorrhagic region. Hemorrhage (light brown within the greenish-yellow region) can be clearly distinguished from viable tissue (dark brown), because the former is always surrounded with non-hemorrhagic infarct tissue (yellow). Thus, the extent of hemorrhage can be quantified.

Tagging Used for Confirming Spatial Correlation and Co-Registration of MRI and TTC Results.

An additional method that we have recently developed for solving the problems of coregistration in comparing the PIM, generated by the methods disclosed, to the gold standard, TTC, is shown in FIG. 21. The end-diastolic (ED) voxel-by-voxel PIM was sectored using the ED grids of the ED tagged cine image, and tag-sector-percent-infarct (TScPI_{PIM}) values were calculated for each such sector by summing the number of voxels and all related PI values in it. The end-systolic (ES) tagging grid was transferred from the ES tagged cine image to the systolic looking TTC slice. In each of these sectors the percentage of TTC-indicated infarcted myocardium was measured, and the tagged-sector-percent infarct (TScPI_{TTC}) value was obtained. TScPI values from PIM and from TTC were correlated pairwise in corresponding sectors. Significant correlation was found between TScPI_{PIM} vs. TScPI_{TTC} values (R=0.8, p<0.01). This method validated the strong spatial correspondence between in-vivo derived PIM (in the presence of PCA) and ex-vivo TTC staining. The fact that R is only 0.8, may be due to the fact that the TTC slices are not perfectly end-systolic, and also that TTC photos only show the surface of each myocardial slice, and some parts of these tortuous infarcts or, on the other hand, some viable areas in the center of the slice, remain undetected. The PIM, generated by the methods disclosed, however, collects information from a 10 mm deep slab. Thus, based on theory, the accuracy of the in-vivo PIM, as disclosed, is better than that of the ex-vivo TTC staining “gold standard”, except there is no in-vivo gold standard to prove this experimentally.

It should be emphasized that the above-described embodiments of the present disclosure are merely possible examples of implementations, and are merely set forth for a clear understanding of the principles of the disclosure. Therefore, many variations and modifications may be made to the above-described embodiment(s) of the disclosure without departing substantially from the spirit and principles disclosed herein. All such modifications and variations are intended to be included herein within the scope of this disclosure and protected by the following claims.

Therefore, the following is claimed:

1. A method, comprising:
obtaining an imaging modality parameter for a plurality of select regions of a structure, wherein each of the measurements of the imaging modality parameter is a partial volume average value of the imaging modality parameter for each of the select regions;
processing the imaging modality parameters for each of the select regions to produce a data set; and
converting the data set into a format to convey information of the data set.

2. The method of claim 1, wherein the format is selected from one of the following: a visual display, a report, a media used to convey information, and a combination thereof.

3. The method of claim 2, further comprising:
forming the visual display of the structure from the imaging modality parameters, wherein the structure is a tissue.

4. The method of claim 1, wherein the structure is a biological material.

5. The method of claim 1, wherein the structure is a non-biological material.

6. The method of claim 1, further comprising:
providing an imaging modality selected from one of the following: magnetic resonance imaging (MRI), SPECT, PET, ultrasound, X-ray imaging, and CAT.

7. The method of claim 1, further comprising:
providing a magnetic resonance imaging (MRI) imaging modality.

8. The method of claim 7, further comprising:
forming a visual display of the structure from the imaging modality parameters, wherein the visual display is selected from one of the following: a parametric map, a functional map, a pathology map, an SI₀ map, and combinations thereof.

9. The method of claim 8, further comprising: determining a clinical parameter based on the visual display.

10. The method of claim 1, further comprising: determining a non-clinical parameter based on the format.

11. A method, comprising:
obtaining a magnetic resonance physical parameter selected from one of R1, R2, R2*, signal intensity (SI), signal intensity at equilibrium (SI₀), proton density (PD), and combinations thereof, for a plurality of select regions of a tissue, wherein R1 is a longitudinal relaxation rate parameter, R2 is the irrecoverable transverse relaxation rate parameter, and R2* the total transverse relaxation rate parameter;
processing the magnetic resonance parameter for each of the select regions; and
forming a visual display of the tissue sample from the magnetic resonance physical parameters.

12. The method of claim 11, further comprising: determining a ΔR1, a ΔR2, a ΔR2*, and combinations thereof, for a plurality of select regions of the tissue.

13. The method of claim 11, further comprising: determining a clinical parameter based on the visual display.

14. The method of claim 13, wherein the clinical parameter is selected from one of the following: tissue pathology, tissue function, a physiological parameter, clinical condition, material imperfection, and combinations thereof.

15. The method of claim 14, wherein the tissue pathology is selected from one of the following: tissue viability, tissue edema, hemorrhage, fibrosis, neoplasm, altered growth rate of tissue, altered volume of tissue, altered density of tissue, altered type of tissue, altered blood flow of tissue, altered tissue perfusion, altered metabolism of tissue, tissue response, and combinations thereof.

16. The method of claim 14, wherein the clinical parameter is a response of the tissue to therapeutic intervention directed to treat a clinical condition of the tissue.

17. The method of claim 14, wherein the clinical parameter is a progression of a clinical condition in the tissue.

18. The method of claim 13, wherein the tissue is myocardial tissue and wherein the clinical parameter is viability.

19. The method of claim 18, wherein the visual display indicates a patchy infarct area in the myocardial tissue.

20. The method of claim 11, wherein the visual display is selected from one of the following: a parametric map, a functional map, a pathology map, an SI₀ map, and combinations thereof.

21. The method of claim 20, wherein the pathology map is a percent pathology map.

22. The method of claim 21, wherein the percent pathology map is a percent infarct map.

23. The method of claim 22, wherein the intrinsic magnetic resonance physical parameter is ΔR1.

24. The method of claim 23, wherein the tissue is myocardial tissue.

25. The method of claim 21, wherein the percent pathology map is a percent edema map.

26. The method of claim 25, wherein the intrinsic magnetic resonance physical parameter is ΔR2.

27. The method of claim 26, wherein the tissue is myocardial tissue.

28. The method of claim 25, further comprising:
generating a tissue characterization map from the percent edema map and a contrast-enhanced T1-weighted image of the tissue sample.

29. The method of claim 28, wherein the tissue characterization map identifies each region of the tissue as a type of tissue selected from the following: healthy tissue, edematous tissue, necrotic tissue, necrotic-hemorrhagic tissue, scar tissue, neoplastic tissue, and combinations thereof.

30. The method of claim 21, wherein the percent pathology map is a percent perfusion map.

31. The method of claim 11, wherein the visual display is three-dimensional.

32. The method of claim 11, further comprising: exposing the tissue to a persistent contrast agent, wherein the persistent contrast agent alters the intrinsic magnetic resonance physical parameter of portions of tissue.

33. A display, comprising a visual display formed by one of the claims 1 to 32.

34. A computer system that implements one or more of the methods of claim 1 to claim 32, comprising a display device that displays a visual display of one of the following: claim 1 to claim 32.

35. A display, comprising:
a visual quantitative display of a tissue sample, wherein the visual display is formed from at least one magnetic resonance physical parameter obtained for each of a set of select regions of the tissue sample, wherein magnetic resonance physical parameter selected from one of R1, R2, R2*, signal intensity (SI), signal intensity at equilibrium (SI₀), proton density (PD), and combinations thereof, wherein R1 is a longitudinal relaxation rate parameter, R2 is the irrecoverable transverse relaxation rate parameter, and R2* is the total transverse relaxation rate parameter.

36. The display of claim 35, wherein the visual display is selected from one of the following: a percent pathology map (PPM), percent infarct map (PIM), percent edema map (PEM), percent perfusion map (PPM), tissue characterization map (TCM) virtual biopsy map (VBM), and combinations thereof.

37. A method of evaluating a condition, comprising:
generating the visual display of claim 25; and
evaluating a clinical parameter illustrated in the visual display, wherein the clinical parameter corresponds to a condition in a tissue.

38. The method of claim 37, further comprising:
diagnosing the condition in the tissue based on the clinical parameter illustrated in the visual display.

39. The method of claim 37, further comprising:
evaluating a treatment for the condition in the tissue based on the clinical parameter illustrated in the visual display.

40. The method of claim 37, further comprising:
planning an effective therapeutic regimen for the condition in the tissue based on the clinical parameter illustrated in the visual display.

41. The method of claim 37, further comprising:
assessing an efficacy of a therapeutic regimen for the condition in the tissue based on the clinical parameter illustrated in the visual display.

42. The method of claim 37, further comprising:
determining the location of the condition in the tissue based on the clinical parameter in the visual display.

43. The method of claim 37, further comprising:
determining the severity of the condition in the tissue based on the clinical parameter illustrated in the visual display.

44. The method of claim 37, wherein the condition is selected from one of the following: altered growth rate of tissues, cancerous transformation of tissues, inflammation or infection of a tissue, altered volume of a tissue, altered density of a tissue, altered blood flow in a tissue, altered physiological function, altered metabolism of a tissue, loss of tissue viability, presence of edema or fibrosis in a tissue, altered perfusion in tissue, and combinations thereof.

45. A display, comprising:
a formatted data set in a format selected from one of the following: a visual display, a report, a media used to convey information, and a combination thereof, wherein the data set is generated from an imaging modality parameter for a plurality of select regions of a structure, wherein each of the measurements of the imaging modality parameter is a partial volume average value of the imaging modality parameter for each of the select regions.

46. The display of claim 45, wherein the structure is a biological material.

47. The display of claim 45, wherein the structure is a non-biological material.

48. The display of claim 45, wherein the imaging modality parameter is a parameter obtained using a technique selected from one of the following: magnetic resonance imaging (MRI), SPECT, PET, ultrasound, X-ray imaging, and CAT.

49. The display of claim 45, wherein the visual display is selected from one of the following: a parametric map, a functional map, a pathology map, an SI₀ map, and combinations thereof.
